SEQUENCE LISTING (159 sequences)

SEQ ID NO: 1 - protein, 736 aa, Unknown
  AAV3B
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 2 - protein, 736 aa, Artificial Sequence
  Synthetic
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQSTASGTTGTSTLRFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQNGRDNPTFRDVQHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 3 - protein, 13 aa, Artificial Sequence
  Synthetic
STASGTTGTSTLR

SEQ ID NO: 4 - protein, 17 aa, Artificial Sequence
  Synthetic
IPGQNNNSNFPWTAAST

SEQ ID NO: 5 - protein, 14 aa, Artificial Sequence
  Synthetic
GKQDTARSDVEVGK

SEQ ID NO: 6 - protein, 14 aa, Artificial Sequence
  Synthetic
QSSNTAPTTRTVND

SEQ ID NO: 7 - protein, 13 aa, Artificial Sequence
  Synthetic
  misc_feature (3)..(3): Xaa can be P or A
  misc_feature (4)..(4): Xaa can be S or G
  misc_feature (10)..(10): Xaa can be S or N
  misc_feature (11)..(11): Xaa can be T or G
  misc_feature (13)..(13): Xaa can be K or R
STXXGTTGTXXLX

SEQ ID NO: 8 - protein, 14 aa, Artificial Sequence
  Synthetic
QNGRDNPTFRDVQH

SEQ ID NO: 9 - protein, 6 aa, Artificial Sequence
  Synthetic
KDDDER

SEQ ID NO: 10 - protein, 736 aa, Artificial Sequence
  Synthetic
  misc_feature (178)..(178): Xaa can be any naturally occurring amino acid
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGXSESVPDPQPLGEPPAAPTSLGSNTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQSTASGTTGTSTLRFSQAGPQSMSLQARNWLPGPCYRQQRLSKIPGQNNNSNFPWTAASTYHLNGRDSLVNPGPAMASHKDDDERFFPMHGNLIFGKQDTARSDVEVGKVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 11 - protein, 17 aa, Artificial Sequence
  Synthetic
TANDNNNSNFPWTAASK

SEQ ID NO: 12 - protein, 6 aa, Artificial Sequence
  Synthetic
KDDEEK

SEQ ID NO: 13 - protein, 12 aa, Artificial Sequence
  Synthetic
EGTTASNAELDN

SEQ ID NO: 14 - protein, 5 aa, Artificial Sequence
  Synthetic
  misc_feature (2)..(2): Xaa can be Q or A
  misc_feature (5)..(5): Xaa can be T or S
SXGAX

SEQ ID NO: 15 - protein, 13 aa, Artificial Sequence
  Synthetic
  misc_feature (1)..(1): Xaa can be G or S
  misc_feature (3)..(3): Xaa can be T, P, or A
  misc_feature (4)..(4): Xaa can be S or G
  misc_feature (8)..(8): Xaa can be N or G
  misc_feature (9)..(9): Xaa can be Q or T
  misc_feature (10)..(10): Xaa can be S or N
  misc_feature (11)..(11): Xaa can be R, T, or G
  misc_feature (13)..(13): Xaa can be L, K, or R
XTXXGTTXXXXLX

SEQ ID NO: 16 - protein, 17 aa, Artificial Sequence
  Synthetic
  misc_feature (1)..(1): Xaa can be I or T
  misc_feature (2)..(2): Xaa can be A or P
  misc_feature (3)..(3): Xaa can be N, S, or G
  misc_feature (4)..(4): Xaa can be D or Q
  misc_feature (17)..(17): Xaa can be K or T
XXXXNNNSNFPWTAASX

SEQ ID NO: 17 - protein, 6 aa, Artificial Sequence
  Synthetic
  misc_feature (4)..(4): Xaa can be E or D
  misc_feature (5)..(5): Xaa can be E or D
  misc_feature (6)..(6): Xaa can be K or R
KDDXXX

SEQ ID NO: 18 - protein, 14 aa, Artificial Sequence
  Synthetic
  misc_feature (3)..(3): Xaa can be E or Q
  misc_feature (4)..(4): Xaa can be G or D
  misc_feature (5)..(5): Xaa can be T or A
  misc_feature (6)..(6): Xaa can be T, A, or G
  misc_feature (7)..(7): Xaa can be A or R
  misc_feature (8)..(8): Xaa can be S or D
  misc_feature (9)..(9): Xaa can be N or D
  misc_feature (10)..(10): Xaa can be A, T, or V
  misc_feature (12)..(12): Xaa can be L, V, or Y
  misc_feature (13)..(13): Xaa can be D or G
  misc_feature (14)..(14): Xaa can be N, K, or H
GKXXXXXXXXEXXX

SEQ ID NO: 19 - protein, 14 aa, Artificial Sequence
  Synthetic
  misc_feature (2)..(2): Xaa can be S or N
  misc_feature (3)..(3): Xaa can be S or G
  misc_feature (4)..(4): Xaa can be N or R
  misc_feature (5)..(5): Xaa can be T or D
  misc_feature (6)..(6): Xaa can be A or N
  misc_feature (9)..(9): Xaa can be T or F
  misc_feature (11)..(11): Xaa can be T or D
  misc_feature (13)..(13): Xaa can be N or Q
  misc_feature (14)..(14): Xaa can be H or D
QXXXXXPTXRXVXX

SEQ ID NO: 20 - DNA, 20 bp, Artificial Sequence
  Synthetic
ggctgggcgacagagtcatc

SEQ ID NO: 21 - DNA, 24 bp, Artificial Sequence
  Synthetic
gctggagatttgcttgtagagatg

SEQ ID NO: 22 - DNA, 60 bp, Artificial Sequence
  Synthetic
catctctacaagcaaatctccagcvvmdcaggagctascaacgacaaccactactttggc

SEQ ID NO: 23 - DNA, 56 bp, Artificial Sequence
  Synthetic
ccaaggaaastyactgttgttgttsysgbygkvgrytkttgaaagtctctgttgcc

SEQ ID NO: 24 - DNA, 52 bp, Artificial Sequence
  Synthetic
aacaacaacagtrastttccttggmcagcggccagcamatatcatctcaatg

SEQ ID NO: 25 - DNA, 58 bp, Artificial Sequence
  Synthetic
gattgccgtgcatagggaaaaatytsycskyatcgtccyygtgactggccatagctgg

SEQ ID NO: 26 - DNA, 23 bp, Artificial Sequence
  Synthetic
atttttccctatgcacggcaatc

SEQ ID NO: 27 - DNA, 22 bp, Artificial Sequence
  Synthetic
catccgtgtgaggaatctttgc

SEQ ID NO: 28 - DNA, 25 bp, Artificial Sequence
  Synthetic
ttgcgttctgttcaggtagtacaga

SEQ ID NO: 29 - DNA, 80 bp, Artificial Sequence
  Synthetic
  misc_feature (33)..(33): n is a, c, g, or t
  misc_feature (62)..(62): n is a, c, g, or t
ctgtactacctgaacagaacgcaargcamcvcnrgcggaacarccrvcmhsmrsvvsctgvngtttagccaggctgggcc

SEQ ID NO: 30 - DNA, 21 bp, Artificial Sequence
  Synthetic
tttgccaaatattagattgcc

SEQ ID NO: 31 - DNA, 80 bp, Artificial Sequence
  Synthetic
cggcaatctaatatttggcaaasaarrcrscrvsrvarvcratrycgmsdwcgrsvrsgtaatgattacggatgaagaag

SEQ ID NO: 32 - DNA, 23 bp, Artificial Sequence
  Synthetic
ctgcaagttatttgccacagttc

SEQ ID NO: 33 - DNA, 78 bp, Artificial Sequence
  Synthetic
  misc_feature (52)..(52): n is a, c, g, or t
gaactgtggcaaataacttgcagrvsvvsmrsrvcvvscccacgdhtvvsrnsgtcvmscatcagggggccttacctg

SEQ ID NO: 34 - DNA, 28 bp, Artificial Sequence
  Synthetic
cagtatctgtactacctgaacagaacgc

SEQ ID NO: 35 - DNA, 28 bp, Artificial Sequence
  Synthetic
gcgttctgttcaggtagtacagatactg

SEQ ID NO: 36 - DNA, 26 bp, Artificial Sequence
  Synthetic
cctgggccctgctaccggcaacagag

SEQ ID NO: 37 - DNA, 26 bp, Artificial Sequence
  Synthetic
ctctgttgccggtagcagggcccagg

SEQ ID NO: 38 - DNA, 28 bp, Artificial Sequence
  Synthetic
ccctatgcacggcaatctaatatttggc

SEQ ID NO: 39 - DNA, 28 bp, Artificial Sequence
  Synthetic
gccaaatattagattgccgtgcataggg

SEQ ID NO: 40 - protein, 496 aa, Unknown
  AAV3B
TTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 41 - DNA, 1491 bp, Unknown
  AAV3B
accaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactggaactgtcaatcatcagggggccttacctggcatggtgtggcaagatcgtgacgtctaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtga

SEQ ID NO: 42 - DNA, 1140 bp, Artificial Sequence
  Synthetic
  misc_feature (636)..(636): n is a, c, g, or t
  misc_feature (665)..(665): n is a, c, g, or t
  misc_feature (1064)..(1064): n is a, c, g, or t
accaccagcaccagaacctgggccctgcccacttacaacaaccatctctacaagcaaatctccagcvvmdcaggagctascaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaargcamcvcnrgcggaacarccrvcmhsmrsvvsctgvngtttagccaggctgggcctcagtctatgtctttgcaggccagaaattggctacctgggccctgctaccggcaacagagactttcaamarycbmcrvcsrsaacaacaacagtrastttccttggmcagcggccagcamatatcatctcaatggccgcgactcgctggtgaatccaggaccagctatggccagtcacrrggacgatrmsgrsaratttttccctatgcacggcaatctaatatttggcaaasaarrcrscrvsrvarvcratrycgmsdwcgrsvrsgtaatgattacggatgaagaagagattcgtaccaccaatcctgtggcaacagagcagtatggaactgtggcaaataacttgcagrvsvvsmrsrvcvvscccacgdhtvvsrnsgtcvmscatcagggggccttacctggcatggtgtggcaagatcgtgacgtctaccttcaaggacctatctgggca

SEQ ID NO: 43 - protein, 620 aa, Artificial Sequence
  Synthetic
  misc_feature (263)..(264): Xaa can be any naturally occurring amino acid
  misc_feature (267)..(267): Xaa can be any naturally occurring amino acid
  misc_feature (450)..(453): Xaa can be any naturally occurring amino acid
  misc_feature (456)..(460): Xaa can be any naturally occurring amino acid
  misc_feature (462)..(462): Xaa can be any naturally occurring amino acid
  misc_feature (491)..(495): Xaa can be any naturally occurring amino acid
  misc_feature (500)..(500): Xaa can be any naturally occurring amino acid
  misc_feature (504)..(504): Xaa can be any naturally occurring amino acid
  misc_feature (508)..(508): Xaa can be any naturally occurring amino acid
  misc_feature (528)..(528): Xaa can be any naturally occurring amino acid
  misc_feature (531)..(533): Xaa can be any naturally occurring amino acid
  misc_feature (546)..(557): Xaa can be any naturally occurring amino acid
  misc_feature (586)..(590): Xaa can be any naturally occurring amino acid
  misc_feature (593)..(595): Xaa can be any naturally occurring amino acid
  misc_feature (597)..(597): Xaa can be any naturally occurring amino acid
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSXXGAXNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQXXXXGTXXXXXLXFSQAGPQSMSLQARNWLPGPCYRQQRLSXXXXXNNNSXFPWXAASXYHLNGRDSLVNPGPAMASHXDDXXXFFPMHGNLIFGKXXXXXXXXXXXXVMITDEEEIRTTNPVATEQYGTVANNLQXXXXXPTXXXVXHQGALPGMVWQDRDVYLQGPIWA

SEQ ID NO: 44 - protein, 49 aa, Unknown
  AAV3B
GTTSGTTNQSRLLKTANDNNNSNFPWTAASKKDDEEKEGTTASNAELDN

SEQ ID NO: 45 - protein, 49 aa, Artificial Sequence
  Synthetic
GNTGGTTSPSRLRKIYDRNNNSNFPWTAASTGDDTGRQGTGEGNVEVGK

SEQ ID NO: 46 - protein, 49 aa, Artificial Sequence
  Synthetic
STTSGTTGQRKLAKAYGHNNNSDFPWPAASTGDDEDRQDSGENDVAIGR

SEQ ID NO: 47 - protein, 49 aa, Artificial Sequence
  Synthetic
GTASGTTANSNLKKTYSDNNNSNFPWTAASKGDDDDREGTDGANVEIDR

SEQ ID NO: 48 - protein, 49 aa, Artificial Sequence
  Synthetic
GTPSGTAAHKTLEKTSAENNNSNFPWTAASKGDDAGREDAEGGDAAIGG

SEQ ID NO: 49 - protein, 49 aa, Artificial Sequence
  Synthetic
STAGGTATQKALTKVHAHNNNSNFPWTAASKEDDTGRQDATRSNVAFEE

SEQ ID NO: 50 - protein, 49 aa, Artificial Sequence
  Synthetic
GNPSGTTGLRGLTTTDNENNNSNFPWPAASKKDDAGKQGTDGNNIAFGE

SEQ ID NO: 51 - protein, 49 aa, Artificial Sequence
  Synthetic
GNTSGTTSKRPLMTTANENNNSNFPWTAASTEDDNEREDAKGTDTEFDR

SEQ ID NO: 52 - protein, 49 aa, Artificial Sequence
  Synthetic
SNASGTTGIHQLKTAPDRNNNSEFPWTAASKEDDNGRQNGATADTEVER

SEQ ID NO: 53 - protein, 49 aa, Artificial Sequence
  Synthetic
SNTGGTTAMRELEKAPNDNNNSKFPWTAASTEDDTGKESTAETDVEDGR

SEQ ID NO: 54 - protein, 49 aa, Artificial Sequence
  Synthetic
SNASGTAGLQRLKKIPDQNNNSNFPWTAASKKDDNGKQSGGAADIDNGN

SEQ ID NO: 55 - protein, 49 aa, Artificial Sequence
  Synthetic
GNPSGTTAPHRLLTIHNGNNNSNFPWPAASTKDDDGRQDGGTSNIDIDG

SEQ ID NO: 56 - protein, 49 aa, Artificial Sequence
  Synthetic
GTPSGTTDLRELAKIPNDNNNSNFPWTAASTEDDEDREGTTASNAELDN

SEQ ID NO: 57 - protein, 49 aa, Artificial Sequence
  Synthetic
SNPSGTANPRTLMKIDAHNNNSEFPWPAASTEDDNGKQSSTTGDVEDDD

SEQ ID NO: 58 - protein, 49 aa, Artificial Sequence
  Synthetic
GNASGTTDTKRLTKASGGNNNSNFPWTAASTKDDDDKEGSNRDDAEVDR

SEQ ID NO: 59 - protein, 49 aa, Artificial Sequence
  Synthetic
GNTGGTTDIRRLRKTHSENNNSEFPWTAASTKDDNERQDTRETDVAIDR

SEQ ID NO: 60 - protein, 49 aa, Artificial Sequence
  Synthetic
GNASGTAGMRELMKAANHNNNSNFPWTAASKEDDEDKESGSADDVAIGR

SEQ ID NO: 61 - protein, 49 aa, Artificial Sequence
  Synthetic
SNTGGTATPKQLQKASAHNNNSEFPWTAASKKDDEEREGSTRNDIANEH

SEQ ID NO: 62 - protein, 49 aa, Artificial Sequence
  Synthetic
SNTGGTANIKELTKTSNDNNNSNFPWTAASTRDDNDKQSASKNDIEYEQ

SEQ ID NO: 63 - protein, 49 aa, Artificial Sequence
  Synthetic
SNAGGTTSNRELRTTSSQNNNSNFPWTAASTRDDDDRQDAGGNDVEVGD

SEQ ID NO: 64 - protein, 49 aa, Artificial Sequence
  Synthetic
SNTSGTATTSALKKTYGHNNNSNFPWTAASKGDDTEKQGGSANNVEVES

SEQ ID NO: 65 - protein, 49 aa, Artificial Sequence
  Synthetic
SNAGGTAATNRLLKIYDRNNNSNFPWTAASTRDDEDKEGGEKGNVDIDR

SEQ ID NO: 66 - protein, 49 aa, Artificial Sequence
  Synthetic
STPSGTTATKGLTTAHTGNNNSNFPWTAASKGDDDGKEGSTATDVAIGS

SEQ ID NO: 67 - protein, 49 aa, Artificial Sequence
  Synthetic
GNTGGTTDLRRLMTTDNHNNNSEFPWPAASTGDDKGRENGAKNDIAFEG

SEQ ID NO: 68 - protein, 49 aa, Artificial Sequence
  Synthetic
STTSGTTTLKALQKIPNRNNNSNFPWTAASTGDDEDRENSKGANTEIDE

SEQ ID NO: 69 - protein, 49 aa, Artificial Sequence
  Synthetic
GTTSGTTDPKDLVTTHGDNNNSDFPWTAASTKDDDDKEDTAADNVEFGR

SEQ ID NO: 70 - protein, 49 aa, Artificial Sequence
  Synthetic
STAGGTTTIKDLVKVPDDNNNSKFPWTAASKEDDDERQDSGATNVEFGR

SEQ ID NO: 71 - protein, 49 aa, Artificial Sequence
  Synthetic
GTTGGTTTMRKLGKVYGGNNNSNFPWTAASTEDDAERQSSGRNDVEYGD

SEQ ID NO: 72 - protein, 49 aa, Artificial Sequence
  Synthetic
GNTGGTASTRRLTKIPDQNNNSNFPWPAASKEDDEGRQSAEKGDIEYGR

SEQ ID NO: 73 - protein, 49 aa, Artificial Sequence
  Synthetic
GNTSGTTATHTLAKIHSRNNNSDFPWPAASKEDDAGKQGATAGNIDVEQ

SEQ ID NO: 74 - protein, 49 aa, Artificial Sequence
  Synthetic
SNPGGTTSIRGLQTIPNRNNNSNFPWPAASTRDDTDKQGGGAGNTDFDH

SEQ ID NO: 75 - protein, 49 aa, Artificial Sequence
  Synthetic
STTSGTAAPRGLVTVYGHNNNSEFPWTAASKKDDEGREGAGASNVAIEE

SEQ ID NO: 76 - protein, 49 aa, Artificial Sequence
  Synthetic
GNASGTATKQGLMKVPNQNNNSDFPWTAASKKDDDDRQSSDKNNAEDDS

SEQ ID NO: 77 - protein, 49 aa, Artificial Sequence
  Synthetic
SNAGGTATTSQLRTAPAENNNSNFPWPAASTRDDADRESGRGDNVDFEK

SEQ ID NO: 78 - protein, 49 aa, Artificial Sequence
  Synthetic
SNTGGTAGIRALQKVDTGNNNSDFPWTAASTEDDTEKENSARNDADIGR

SEQ ID NO: 79 - protein, 49 aa, Artificial Sequence
  Synthetic
GNASGTAAQNGLRKIPNENNNSKFPWTAASTKDDEGKESSSGDDAEFGG

SEQ ID NO: 80 - protein, 49 aa, Artificial Sequence
  Synthetic
SNTSGTAGPQQLRKTHAQNNNSNFPWTAASKEDDTGKEGARANDIAFDQ

SEQ ID NO: 81 - protein, 49 aa, Artificial Sequence
  Synthetic
STPSGTTSMRTLEKAPARNNNSEFPWTAASTRDDAGKQGSRENDTEFDG

SEQ ID NO: 82 - protein, 49 aa, Artificial Sequence
  Synthetic
STAGGTTALKGLKTIADHNNNSEFPWPAASTRDDKEKEDSTGANIADDR

SEQ ID NO: 83 - protein, 49 aa, Artificial Sequence
  Synthetic
STPSGTASTRTLMKTHNHNNNSEFPWTAASTKDDEDKEGTEATNVAIGG

SEQ ID NO: 84 - protein, 49 aa, Artificial Sequence
  Synthetic
GNPGGTTNNQALRKIHGQNNNSDFPWTAASTRDDNDKEGSARGDVAYEK

SEQ ID NO: 85 - protein, 49 aa, Artificial Sequence
  Synthetic
SNASGTTDTRELVTIADDNNNSEFPWTAASTRDDTDKQGSAGADVEVEK

SEQ ID NO: 86 - protein, 49 aa, Artificial Sequence
  Synthetic
SNPGGTTNLRELRTIHTENNNSEFPWTAASTRDDKDRQGGGGTNVEIGS

SEQ ID NO: 87 - protein, 49 aa, Artificial Sequence
  Synthetic
GTTGGTANNNTLLKIASGNNNSNFPWTAASTKDDKGREGAEKNDTAVGN

SEQ ID NO: 88 - protein, 49 aa, Artificial Sequence
  Synthetic
GTTSGTTDKQQLMKTHNGNNNSDFPWTAASKEDDTGKQSAEGNNVAYDG

SEQ ID NO: 89 - protein, 49 aa, Artificial Sequence
  Synthetic
GTAGGTATLSTLVKISAGNNNSDFPWTAASKGDDNGKQNSTASDVAIDG

SEQ ID NO: 90 - protein, 49 aa, Artificial Sequence
  Synthetic
SNTSGTAGLRTLTKADADNNNSDFPWPAASTGDDNGKEDASGNNVEDGR

SEQ ID NO: 91 - protein, 49 aa, Artificial Sequence
  Synthetic
SNASGTATPSTLRKTDTHNNNSEFPWPAASKRDDNDREGARGSNIDVGD

SEQ ID NO: 92 - protein, 49 aa, Artificial Sequence
  Synthetic
STASGTASLRALMKVPNRNNNSNFPWTAASTGDDNDKENARASDAEVDR

SEQ ID NO: 93 - protein, 49 aa, Artificial Sequence
  Synthetic
GTASGTATTKGLLKISTQNNNSNFPWTAASKEDDADKQGGETDNVDVGD

SEQ ID NO: 94 - protein, 49 aa, Artificial Sequence
  Synthetic
GTASGTAALKQLAKADSDNNNSNFPWTAASTEDDADRQGGETGNIEYDG

SEQ ID NO: 95 - protein, 49 aa, Artificial Sequence
  Synthetic
GNASGTATTSNLMKADDRNNNSNFPWTAASTKDDEDRQGAKRSDTAVEE

SEQ ID NO: 96 - protein, 49 aa, Artificial Sequence
  Synthetic
GTAGGTANMKDLRTTASENNNSDFPWTAASTEDDKDKEGANGGDVAIGQ

SEQ ID NO: 97 - protein, 49 aa, Artificial Sequence
  Synthetic
STPGGTTTIRDLKTVSTDNNNSDFPWPAASTKDDDDREGSGRNNVAVEE

SEQ ID NO: 98 - protein, 49 aa, Artificial Sequence
  Synthetic
STPSGTANINTLRKTPNRNNNSNFPWTAASTEDDEGRQSATKDDVDIGG

SEQ ID NO: 99 - protein, 49 aa, Artificial Sequence
  Synthetic
GNTGGTAGLQKLMKTHGGNNNSNFPWPAASTKDDDGKQSSRGNDVAVDD

SEQ ID NO: 100 - protein, 49 aa, Artificial Sequence
  Synthetic
GNTSGTTTPRTLAKIPSHNNNSEFPWTAASKKDDEEKQGSNGSNIEFGS

SEQ ID NO: 101 - protein, 49 aa, Artificial Sequence
  Synthetic
SNAGGTAGLRQLTKAPAENNNSDFPWTAASTEDDAGKEGGGGANIAVEE

SEQ ID NO: 102 - protein, 49 aa, Artificial Sequence
  Synthetic
GTASGTAAKSTLVKISTRNNNSNFPWTAASTEDDEEKEGTSKNDVEVEN

SEQ ID NO: 103 - protein, 49 aa, Artificial Sequence
  Synthetic
GTTSGTTTTRRLMKIYGGNNNSNFPWTAASTEDDAGRQGTATANVEVES

SEQ ID NO: 104 - protein, 49 aa, Artificial Sequence
  Synthetic
GTAGGTAGMRELATIYTGNNNSNFPWTAASKKDDEGREGSSTGDADVGR

SEQ ID NO: 105 - protein, 49 aa, Artificial Sequence
  Synthetic
STTGGTANPKELRTAANHNNNSEFPWTAASTEDDEDKQGAGESNVAIDG

SEQ ID NO: 106 - protein, 49 aa, Artificial Sequence
  Synthetic
GTTSGTTGTSTLRKTDTGNNNSNFPWTAASKGDDEEREGAGTADAAVDG

SEQ ID NO: 107 - protein, 49 aa, Artificial Sequence
  Synthetic
GNAGGTTNKRDLLTAYTRNNNSDFPWTAASTKDDDEKQGTGKTDADNGG

SEQ ID NO: 108 - protein, 49 aa, Artificial Sequence
  Synthetic
GNASGTTDMKHLTKISDRNNNSNFPWPAASTRDDNERQSTRGGNAEIDG

SEQ ID NO: 109 - protein, 49 aa, Artificial Sequence
  Synthetic
SNTGGTADLRDLLTIPTQNNNSEFPWTAASKKDDNDRQSAKANDVEVDR

SEQ ID NO: 110 - protein, 49 aa, Artificial Sequence
  Synthetic
STASGTTATQQLVTTDSQNNNSEFPWTAASTRDDNDRENAEGGNVEIGQ

SEQ ID NO: 111 - protein, 49 aa, Artificial Sequence
  Synthetic
GNAGGTTANKTLMKIAAHNNNSEFPWTAASTRDDDGKQDSSADNIEYGK

SEQ ID NO: 112 - protein, 49 aa, Artificial Sequence
  Synthetic
GNAGGTTGTKELRTIANENNNSNFPWTAASTKDDDEKEDAKRNNVDYDG

SEQ ID NO: 113 - protein, 49 aa, Artificial Sequence
  Synthetic
SNPSGTTGKSSLKKTSNENNNSNFPWTAASKKDDNGKEDSRAGDADFEK

SEQ ID NO: 114 - protein, 49 aa, Artificial Sequence
  Synthetic
GNTSGTASIRQLQKTPDGNNNSKFPWTAASKRDDNGREGSTEGNIEIEG

SEQ ID NO: 115 - protein, 49 aa, Artificial Sequence
  Synthetic
GNTGGTTTLSALGTAHTQNNNSNFPWTAASKRDDNDKQSSTGGDTAFDG

SEQ ID NO: 116 - protein, 49 aa, Artificial Sequence
  Synthetic
SNPSGTTTTQRLQKTDNDNNNSDFPWPAASTRDDTDREGSTGGDAEIER

SEQ ID NO: 117 - protein, 49 aa, Artificial Sequence
  Synthetic
SNASGTTTMRKLGTISSGNNNSNFPWPAASKGDDNEKQDSSENDVADER

SEQ ID NO: 118 - protein, 49 aa, Artificial Sequence
  Synthetic
STAGGTATMQRLMTTDTGNNNSNFPWTAASTRDDNERQGGEGGDIEDDR

SEQ ID NO: 119 - protein, 49 aa, Artificial Sequence
  Synthetic
GTAGGTATTRDLQTTDDHNNNSDFPWTAASKKDDNGRQGGRGANTAYEG

SEQ ID NO: 120 - protein, 49 aa, Artificial Sequence
  Synthetic
GTTGGTAAMSALRTTDDGNNNSKFPWPAASKKDDEEKEDGGTSNAAIGD

SEQ ID NO: 121 - protein, 49 aa, Artificial Sequence
  Synthetic
GNTSGTTTNRELMKIPNDNNNSNFPWTAASTRDDEDKEDTGRADVEVGR

SEQ ID NO: 122 - protein, 49 aa, Artificial Sequence
  Synthetic
SNAGGTADKQDLVTAHSENNNSNFPWTAASKEDDDDRQGAAGGDIEVGS

SEQ ID NO: 123 - protein, 49 aa, Artificial Sequence
  Synthetic
GNASGTAATHELLTTHDHNNNSDFPWTAASKRDDAEREGGAKSDVDFGS

SEQ ID NO: 124 - protein, 49 aa, Artificial Sequence
  Synthetic
SNASGTADTRHLMTTPGENNNSDFPWPAASTGDDTGKQGSATTDIEYGE

SEQ ID NO: 125 - protein, 49 aa, Artificial Sequence
  Synthetic
SNTSGTTAQSKLQKIHNRNNNSDFPWTAASTRDDDDRQGAEGSDVAVGD

SEQ ID NO: 126 - protein, 49 aa, Artificial Sequence
  Synthetic
GNPSGTADQRALQKIPTGNNNSDFPWTAASTRDDTEKQGTGGSDIEIGG

SEQ ID NO: 127 - protein, 49 aa, Artificial Sequence
  Synthetic
SNASGTAGLNALKKAYTHNNNSDFPWTAASTGDDDERENAKAGNAAIDG

SEQ ID NO: 128 - protein, 49 aa, Artificial Sequence
  Synthetic
GNPSGTTNLQRLMKIDDQNNNSNFPWPAASTKDDEDKESGGTADVAVDK

SEQ ID NO: 129 - protein, 49 aa, Artificial Sequence
  Synthetic
GTTSGTASIQRLQKTYADNNNSEFPWTAASTKDDEDKESAAGSDTEVDG

SEQ ID NO: 130 - protein, 12 aa, Artificial Sequence
  Synthetic
QNKTSRHPDEAG

SEQ ID NO: 131 - protein, 6 aa, Artificial Sequence
  Synthetic
NTSDAG

SEQ ID NO: 132 - protein, 9 aa, Artificial Sequence
  Synthetic
QHPLKNTMI

SEQ ID NO: 133 - protein, 6 aa, Artificial Sequence
  Synthetic
SQHRKN

SEQ ID NO: 134 - protein, 12 aa, Artificial Sequence
  Synthetic
RKNTSQHPEDAG

SEQ ID NO: 135 - protein, 11 aa, Artificial Sequence
  Synthetic
LKTRMQPEAGV

SEQ ID NO: 136 - protein, 4 aa, Artificial Sequence
  Synthetic
TIAV

SEQ ID NO: 137 - protein, 6 aa, Artificial Sequence
  Synthetic
APHDSY

SEQ ID NO: 138 - protein, 6 aa, Artificial Sequence
  Synthetic
NTSDAG

SEQ ID NO: 139 - protein, 6 aa, Artificial Sequence
  Synthetic
DEGQHR

SEQ ID NO: 140 - protein, 4 aa, Artificial Sequence
  Synthetic
NKED

SEQ ID NO: 141 - protein, 4 aa, Artificial Sequence
  Synthetic
KREG

SEQ ID NO: 142 - protein, 6 aa, Artificial Sequence
  Synthetic
ETKNAD

SEQ ID NO: 143 - protein, 4 aa, Artificial Sequence
  Synthetic
GNSD

SEQ ID NO: 144 - protein, 4 aa, Artificial Sequence
  Synthetic
TSGA

SEQ ID NO: 145 - protein, 9 aa, Artificial Sequence
  Synthetic
TKNRSEDAG

SEQ ID NO: 146 - protein, 6 aa, Artificial Sequence
  Synthetic
AKTREG

SEQ ID NO: 147 - protein, 6 aa, Artificial Sequence
  Synthetic
SNTDAG

SEQ ID NO: 148 - protein, 4 aa, Artificial Sequence
  Synthetic
ATIV

SEQ ID NO: 149 - protein, 7 aa, Artificial Sequence
  Synthetic
LNIDVYF

SEQ ID NO: 150 - protein, 9 aa, Artificial Sequence
  Synthetic
NKRSQHEDG

SEQ ID NO: 151 - protein, 9 aa, Artificial Sequence
  Synthetic
SKNTREDAG

SEQ ID NO: 152 - protein, 12 aa, Artificial Sequence
  Synthetic
SKNTRQHPEDAG

SEQ ID NO: 153 - protein, 6 aa, Artificial Sequence
  Synthetic
NQHRKS

SEQ ID NO: 154 - protein, 6 aa, Artificial Sequence
  Synthetic
TNSDAG

SEQ ID NO: 155 - protein, 12 aa, Artificial Sequence
  Synthetic
AKNTRSQHPEDG

SEQ ID NO: 156 - protein, 9 aa, Artificial Sequence
  Synthetic
TNIDAVYSF

SEQ ID NO: 157 - protein, 12 aa, Artificial Sequence
  Synthetic
GKNTRSQHPEDA

SEQ ID NO: 158 - protein, 12 aa, Artificial Sequence
  Synthetic
TKNRSMIEDAGV

SEQ ID NO: 159 - protein, 9 aa, Artificial Sequence
  Synthetic
NTKPQHAED

(19) United States
(12) Patent Application Publication
(10) Pub. No.: US 2023/0049066 A1
(43) Pub. Date: Feb. 16, 2023

(54) NOVEL AAV3B VARIANTS THAT TARGET HUMAN HEPATOCYTES IN THE LIVER OF HUMANIZED MICE

(71) Applicants: University of Florida Research Foundation, Incorporated, Gainesville, FL (US); University of Massachusetts One Beacon Street, Boston, MA (US)

(72) Inventors: Sergei Zolotukhin, Gainesville, FL (US); Damien Marsic, Gainesville, FL (US); Christian Murller, Concord, MA (US)

(73) Assignees: University of Florida Research Foundation, Incorporated, Gainesville, FL (US); University of Massachusetts One Beacon Street, Boston, FL (US)

(21) Appl. No.: 17/779,505

(22) PCT Filed: Nov. 24, 2020
(86) PCT No.: PCT/US2020/062114

§ 371 Date: May 24, 2022

Related U.S. Application Data
(60) Provisional application No. 62/940,162, filed on Nov. 25, 2019.

(51) Int. Cl. A61K 48/00; C12N 15/86; C07K 7/08

(52) U.S. Cl. CPC A61K 48/0025; C12N 15/86; C07K 7/08; C12N 2750/14122; C12N 2750/14123; C12N 2750/14142; C12N 2750/14145; A61K 38/00

(57) ABSTRACT

Disclosed herein are recombinant AAV variant (e.g., variant serotype 3B (AAV3B)) capsid proteins and variant capsid protein-containing viral particles with enhanced ability to transduce hepatic cells. Viral particles containing these capsid variants are capable of evading neutralization by the host humoral immune response. The recombinant AAV3B variant proteins and viral particles disclosed herein were identified from a variant AAV3B capsid library that was engineered by making substitutions in only the variable regions of the capsid. Some embodiments of the AAV3B capsid variants disclosed herein comprise the AAV3B-G3 variant and the AAV3B-E12 variant. Compositions of these variant AAV particles are provided that are useful for transducing and delivering therapeutic transgenes to cells, such as liver cells, and thus treat diseases and disorders pertaining to these cells.

### RELATED APPLICATIONS

This application claims the benefit under 35 U.S.C. § 119(e) of the filing date of U.S. Provisional Application Ser. No. 62/940,162, filed Nov. 25, 2019, the entire contents of which are incorporated herein by reference.

### GOVERNMENT SUPPORT

The invention was made with government support under Grant No. HL097088 awarded by the National Institutes of Health. The government has certain rights in the invention.

### BACKGROUND OF THE INVENTION

Adeno-associated virus (AAV) is a single-stranded DNA virus belonging to the Parvoviridae family (Muzyczka and Berns, 2001). AAV-derived vectors are promising tools for human gene therapy applications because of their absence of pathogenicity, low immunogenicity, episomal localization and stable transgene expression. However, significant limitations to the clinical use of AAV are its promiscuity and its susceptibility to neutralization by human antibodies (Jeune et al., 2013). Both of these limitations are determined by nature of the amino acid residues exposed at the surface of the capsid. Therefore, major efforts aiming at developing useful and effective gene therapy vectors have been devoted to obtaining and studying capsid variants (Wu et al., 2006). The first approach was to study naturally occurring AAV isolates. So far, 13 serotypes have been formally characterized and hundreds of variant isolates have been sequenced. Additional capsid variation has been investigated through the generation of mosaics (viral particles made of capsid proteins from more than one serotype) (Hauck et al., 2003; Stachler and Bartlett, 2006; Gigout et al., 2005), chimeras (capsid proteins with domains from various origins) (Shen et al., 2007), and various substitutional or insertional mutants (Wu et al., 2000). However, the most significant advances are expected to result from directed evolution approaches through the development of capsid libraries.

The state of the art method for randomizing an AAV capsid-encoding genetic sequence was until recently error-prone polymerase chain reaction (PCR), which introduced randomly dispersed mutations throughout the roughly 730 amino acids that constitute the AAV capsid sequence. However, the error-prone PCR technique suffered from two key problems. First, random mutagenesis often installed mutations that were deleterious to capsid function, as only 0.01-1% of mutations were typically beneficial (see, e.g., Romero & Arnold (2009), Nat Rev Mol Cell Biol 10(12): 866-876 and Guo, Choe & Loeb (2004), Proc Natl Acad Sci USA 101(25): 9205-9210.). Second, the sheer number of PCR clones that needed to be generated to cover all combinations of multiple mutations within a single capsid by far exceeded the technical capabilities of the skilled artisan. For instance, it has been calculated that, to comprehensively randomize five residues within a 414 base pair fragment of the AAV2 capsid VP1 gene, an AAV library would have to comprise nearly 10¹¹ different clones to cover a single mutation at each site (see Maersch et al. (2010), Virology 397(1): 167-175).

The various strategies to generate capsid libraries that have been developed so far all suffer from sequence bias or limited diversity. Random display peptide libraries (Govindasamy et al., 2006) are limited to an insertion at one particular capsid location. Libraries generated using error-prone PCR contain a very small fraction of gene variants encoding proteins that can fold properly and assemble into a functional capsid, due to the randomness of the mutations. DNA shuffling and staggered extension processes are more efficient because they recombine naturally-occurring parental sequences and therefore are more likely to generate actual capsid variants. However, they can only recombine blocks of DNA as opposed to single nucleotide positions, which results in sequence bias (parental polymorphisms will tend to cluster together instead of being randomly distributed).

### SUMMARY OF THE INVENTION

The present disclosure provides adeno-associated virus (AAV) capsid variants and virions comprising capsid variants that exhibit enhanced transduction in hepatocytes. In certain embodiments, these capsid variants are capable of evading neutralization by host antibodies.

Accordingly, in some aspects, the present disclosure provides modified capsids of serotype 3B, also known as modified AAV3B capsids or AAV3B variants. In some aspects, the present disclosure provides the AAV3B-G3 capsid variant, or “G3.” In some aspects, the present disclosure provides the AAV3B-E12 capsid variant, or “E12.” G3 contains 15 amino acid substitutions, and E12 contains 24 substitutions, relative to wild-type AAV3B.

The present disclosure is based, at least in part, on the rational generation of an AAV capsid variant library through the introduction of motifs of novel mutations in the native capsid through mutagenesis and directed evolution. According to the present disclosure, molecular evolution using a combinatorial library platform has generated capsid variants with high hepatocyte tropism.

The development of next-generation rAAV viral particles, or virions, may dramatically reduce the number of viral particles needed for a conventional gene therapy regimen. In addition to having improved transduction efficiencies for various mammalian cells, the rAAV virions prepared as described herein may be more stable, less immunogenic, safer, and/or may be produced at much lower cost, or in a higher titer, than an equivalent wild type viral vector prepared in conventional fashion. Thus, the increase in targeting efficiency and specificity conferred by the mutations in the motifs of the disclosed capsid variants may improve the safety and therapeutic efficacy, and reduce the production cost, of the associated rAAV treatment.

In the practice of the present disclosure, native amino acids normally present in the sequence of a viral capsid protein, such as a wild-type capsid of serotype 3B, may be substituted by one or more non-native amino acids, including substitutions of one or more amino acids not normally present at a particular residue in the corresponding wild-type protein.

In some embodiments, the amino acid substitutions in the disclosed capsid variants may be epistatic (interacting) with respect to one another. These amino acid substitutions may act synergistically on capsid binding and transduction behavior. In some embodiments, the amino acid substitutions comprise one or more motifs.

In some embodiments, the amino acid substitutions in the disclosed capsid variants confer upon virions comprising these variants an enhanced ability to evade neutralizing antibodies of the host immune system. In some embodiments, the disclosed virions are able to evade the humoral immune response, e.g. neutralizing antibodies, of a subject following their delivery into the subject. In particular embodiments, the subject is mammalian. The subject may be human. The subject may be a non-human primate.

Rationally-generated AAV capsid libraries containing modified AAV capsids based on serotype 3b have been recently described. See International Patent Publication No. WO 2017/070476, published on Apr. 27, 2017, herein incorporated by reference. AAV3B capsid variants containing various combinations of mutations in the surface-exposed Y, S, and T residues have been generated, and an S633V+T492V mutant (AAV3B.ST, or AAV3-ST) was identified to possess enhanced capacity to transduce primary human hepatocytes in vitro. See Ling, C, et al., Mol Ther. 2014; 22: S2, incorporated herein by reference. Unlike what would be expected from error-prone PCR, the novel mutations of the present disclosure were not randomly or arbitrarily selected.

The present disclosure is further based on the screening of variants from amongst the AAV3B library. An iterative, multi-round selection process was performed by injecting the original master AAV3B library for the first round and target-enriched libraries in subsequent rounds. Target-enriched libraries were then generated, purified and quantified. After multiple rounds of screening of this library for enhanced transduction efficiencies in human liver tissue engrafted onto transgenic mice, the modified capsids of the present disclosure were selected.

Certain embodiments of the modified AAV capsids and AAV virions of the present disclosure include the second nucleotide sequence encoding an AAV Cap protein that differs from wildtype serotype 3 VP1 capsid protein at least at one amino position. The at least one amino acid position that differs is preferably in a variable region (VR), and may be in variable regions 1, 4, 5, 6, 7, or 8 (VR-I, VR-IV, VR-V, VR-VI, VR-VII, VR-VIII) and combinations thereof. See FIG. 10.

The present disclosure provides variant recombinant adeno-associated virus (rAAV) serotype 3B (AAV3B) capsid protein comprising any of the following sets of sequences and/or substitutions in the wild-type of AAV3B VP1 sequence of SEQ ID NO: 1. Certain aspects of the modified AAV capsids and AAV virions of the present disclosure include VR-I encoding amino acid sequence SX₁GAX₂ (SEQ ID NO: 14) where X₁ is independently Q or A and X₂ is independently T or S. In certain embodiments, X₁ is A and X₂ is S.

Certain aspects of the modified AAV capsids and AAV virions of the present disclosure include VR-IV encoding amino acid sequence X₃TX₄X₅GTTX₆X₇X₈X₉LX₁₀ (SEQ ID NO: 15) where X₃ is independently G or S; X₄ is independently T, P or A; X₅ is independently S or G; X₆ is independently N or G; X₇ is independently Q or T; X₈ is independently S or N; X₉ is independently R, T or G; and X₁₀ is independently L, K or R. In certain embodiments, X₃ is 5; X₄ is P; X₅ is G; X₆ is G; X₇ is T; X₈ is N; X₉ is G; and X₁₀ is K. In some embodiments, the VR-IV encodes the sequence STX₄X₅GTTGTX₈X₉LX₁₀ (SEQ ID NO: 7), where X₄ is independently P or A; X₈ is independently S or G; X₈ is independently S or N; X₉ is independently T or G; and X₁₀ is independently K or R. In certain embodiments, the VR-IV of the modified capsid encodes an amino acid sequence motif of STASGTTGTSTLR (SEQ ID NO: 3).

Certain aspects of the modified AAV capsids and AAV virions of the present disclosure include VR-V encoding amino acid sequence X₁₁X₁₂X₁₃X₁₄NNNSNFPWTAASX₁₅ (SEQ ID NO: 16) where X₁₁ is independently I or T; X₁₂ is independently A or P; X₁₃ is independently N, S or G; X₁₄ is independently D or Q; and X₁₅ is independently K or T. In certain embodiments, X₁₁ is I; X₁₂ is P; X₁₃ is 5; X₁₄ is Q; and X₁₅ is K. In certain embodiments, the VR-V of the modified capsid encodes an amino acid sequence motif of IPGQNNNSNFPWTAAST (SEQ ID NO: 4). In certain embodiments, the VR-V of the modified capsid encodes an amino acid sequence motif of TANDNNNSNFPWTAASK (SEQ ID NO: 11).

Certain aspects of the modified AAV capsids and AAV virions of the present disclosure include VR-VI encoding amino acid sequence KDDX₁₆X₁₇X₁₈ (SEQ ID NO: 17) where X₁₆ is independently E or D; X₁₇ is independently E or D; and X₁₈ is independently K or R. In certain embodiments, X₁₆ is D; X₁₇ is D; and X₁₈ is R. In certain embodiments, the VR-VI of the modified capsid encodes KDDDER (SEQ ID NO: 9). In certain embodiments, the VR-VI of the modified capsid encodes KDDEEK (SEQ ID NO: 12).

Certain aspects of the modified AAV capsids and AAV virions of the present disclosure include VR-VII encoding amino acid sequence GKX₁₉X₂₀X₂₁X₂₂X₂₃X₂₄X₂₅X₂₆EX₂₇X₂₈X₂₉ (SEQ ID NO: 18) where X₁₉ is independently E or Q; X₂₀ is independently G or D; X₂₁ is independently T or A; X₂₂ is independently T, A or G; X₂₃ is independently A or R; X₂₄ is independently S or D; X₂₅ is independently N or D; X₂₆ is independently A, T or V; X₂₇ is independently L, V or Y; X₂₈ is independently D or G; and X₂₉ is independently N, K or H. In certain embodiments, X₁₉ is Q; X₂₀ is G; X₂₁ is A; X₂₂ is G; X₂₃ is R; X₂₄ is D; X₂₅ is N; X₂₆ is T; X₂₇ is Y; X₂₈ is D; and X₂₉ is H. In certain embodiments, the VR-VII of the modified capsid encodes an amino acid sequence motif of GKQDTARSDVEVGK (SEQ ID NO: 5). In other embodiments, the VR-VII of the modified capsid encodes an amino acid sequence motif of EGTTASNAELDN (SEQ ID NO: 13).

Certain aspects of the modified AAV capsids and AAV virions of the present disclosure include VR-VIII encoding amino acid sequence QX₃₀X₃₁X₃₂X₃₃X₃₄PTX₃₅RX₃₆VX₃₇X₃₈ (SEQ ID NO: 19) where X₃₀ is independently S or N; X₃₁ is independently S or G; X₃₂ is independently N or R; X₃₃ is independently T or D; X₃₄ is independently A or N; X₃₅ is independently T or F; X₃₆ is independently T or D; X₃₇ is independently N or Q; and X₃₈ is independently H or D. In certain embodiments, X₃₀ is 5; X₃₁ is 5; X₃₂ is N; X₃₃ is T; X₃₄ is A; X₃₅ is F; X₃₆ is T; X₃₇ is N; and X₃₈ is D. In certain embodiments, the VR-VIII of the modified capsid encodes an amino acid sequence motif of QSSNTAPTTRTVND (SEQ ID NO: 6). In other embodiments, the VR-VIII of the modified capsid encodes an amino acid sequence motif of QNGRDNPTFRDVQH (SEQ ID NO: 8).

In some embodiments, the AAV virions of the present disclosure are incorporated into at least one host cell. Examples of suitable host cells are mammalian cells including human host cells, including, for example, blood cells, stem cells, hematopoietic cells, CD34 cells, liver cells, cancer cells, vascular cells, pancreatic cells, neural cells, ocular or retinal cells, epithelial or endothelial cells, dendritic cells, fibroblasts, or any other cell of mammalian origin, including, without limitation, hepatic (i.e., liver) cells, lung cells, cardiac cells, pancreatic cells, intestinal cells, diaphragmatic cells, renal (i.e., kidney) cells, neural cells, blood cells, bone marrow cells, or any one or more selected tissues of a mammal for which viral-based gene therapy is contemplated. In some embodiments, the host cells are liver cells (hepatocytes). In some embodiments, the host cells are human liver cells.

AAV virions comprising the exemplary AAV3B variants of the present disclosure may include the virions as incorporated or transduced into at least one host cell. Examples of suitable host cells include human hepatocytes, e.g. hepatocellular carcinoma cell lines HUH-7 and HepG2, murine hepatocytes, e.g. H2.35, HEK293 (embryonic kidney) cells, HeLa cells, Cos cells, U87 cells, KB cells, and Vero cells. In certain embodiments, the modified AAV3B virions of the present disclosure are incorporated into HUH-7, H2.35 and/or HepG2 cells. In particular embodiments, virions comprising the E12 and G3 variants are incorporated into HUH-7, H2.35 and/or HepG2 cells.

In some embodiments, the AAV virions of the present disclosure further comprise a nucleotide sequence encoding at least one molecule providing helper function. The third nucleotide sequence may be a polynucleotide derived from an adenovirus or a herpes virus (e.g. HSV1). In particular embodiments, the polynucleotide is derived from adenovirus, e.g. Ad5.

In some aspects, the disclosure provides methods of selecting tissue-specific or cell-specific variants of an AAV virion includes (a) introducing a plurality of AAV virions into target tissues or cells; (b) allowing sufficient time to elapse to propagate additional virions; and (c) isolating the virions. Steps (a) through (c) may be repeated one or more times to enrich for a tissue-specific (e.g., hepatic tissue-specific) or cell-specific variant. Such enriched variants exhibit a higher target tropism for the target tissues or cells as compared to AAV serotype 3.

An embodiment of the AAV virions of the present disclosure includes (a) a first nucleotide sequence encoding at least one therapeutic molecule; (b) a second nucleotide sequence comprising a regulatory sequence; (c) a third nucleotide sequence comprising a first AAV terminal repeat (e.g., from serotype 3); (d) a fourth nucleotide sequence comprising a second AAV terminal repeat (e.g., from serotype 3); and (e) a capsid comprising at least one AAV Cap protein that differs from wildtype serotype 3 at least at one amino acid position. The first nucleotide sequence is operably linked to the second nucleotide sequence and the first and second nucleotide sequences are interposed between the first and second AAV terminal repeat to form a transgene, and the resulting transgene is packaged within the capsid. Examples of suitable regulatory sequences include promoters and enhancers, e.g., a tissue specific promoter. Examples of suitable therapeutic molecules include polypeptides, peptides, antibodies, antigen binding fragments, growth factors, cytokines and other small therapeutic proteins, and any combination thereof.

In some aspects, the present disclosure provides methods for treating a disease or disorder. Such methods may comprise administering an effective amount of an AAV virion of the present disclosure. In some embodiments, the disease or disorder is Alpha-1 Antitrypsin Deficiency or Transthyretin-Related Familial Amyloid Polyneuropathy.

The following drawings form part of the present specification and are included to demonstrate certain aspects of the present invention. The invention may be better understood by reference to the following description taken in conjunction with the accompanying drawings, in which like reference numerals identify like elements.

### BRIEF DESCRIPTION OF THE DRAWINGS

FIG. 1 shows the wildtype (WT) nucleotide sequence (bottom rows) and corresponding WT amino acids (top rows, bold font) of AAV3B capsid gene and capsid protein, respectively. Degenerate positions within each variable region (VR) diversified in AAV serotype 3 capsid library (A3CL) are highlighted. The degenerate nucleotide positions (in IUPAC code) encoded by synthetic oligonucleotides are shown in italics below the WT sequence.

FIG. 2 shows the nucleotide sequence of the synthetic fragment A3CL as designed. The degenerate nucleotide positions (in IUPAC code) are underlined. The overlap stretches of the synthetic DNA and the plasmid vector backbone are highlighted.

FIG. 3 shows the amino acid sequence of AAV3B VP1. Degenerate positions are labeled by X and underlined.

FIG. 4 shows the amino acid sequences of the A3CL VRs encompassing WT AAV3B VP1 capsid residues 259-600. WT sequences are shown in black, degenerate residues—in italics. Not modified conservative residues between VRs are not shown. VRs borders are indicated by vertical lines.

FIG. 5 is a flowchart illustrating design and construction of AAV3B (A3CL) combinatorial capsid libraries ABC and D.

FIGS. 6-9 are photographs of agarose gels showing products of PCR reactions as per Example 2.

FIG. 10 shows an amino acid sequence alignment between wild-type AAV3B VP1 region and the AAV3B-G3 and AAV3B-E12 variants.

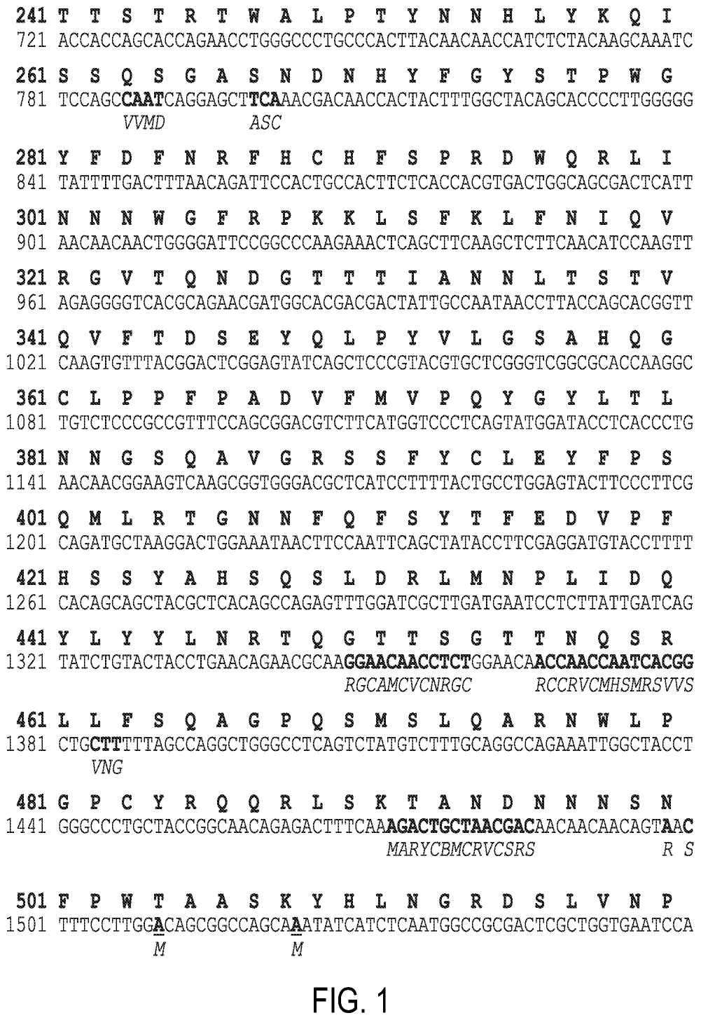

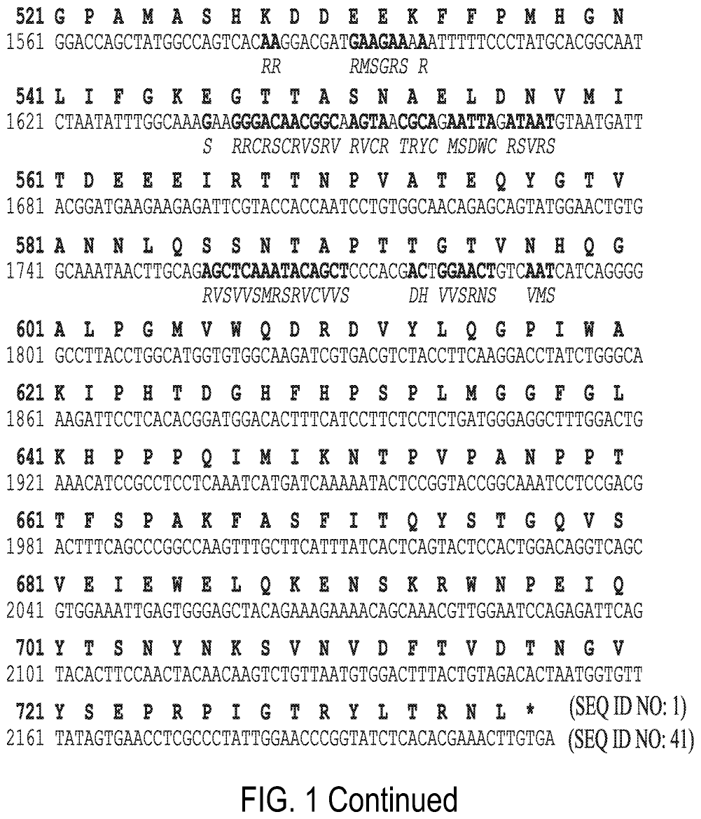

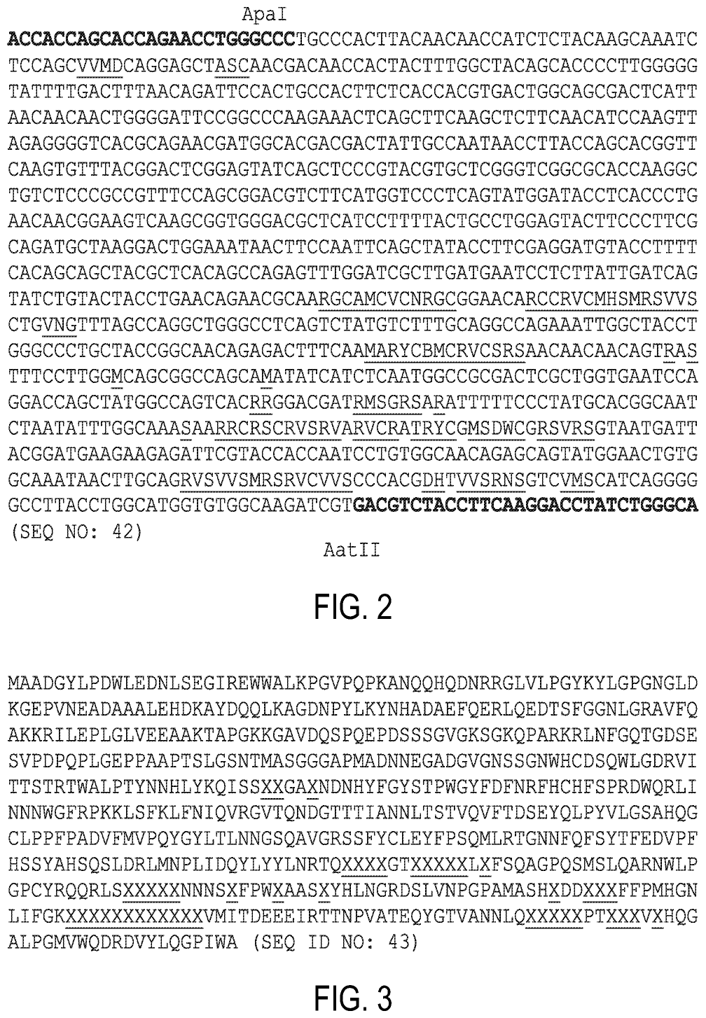

### DETAILED DESCRIPTION OF THE INVENTION

AAV-derived viral particles are promising tools for human gene therapy applications because of reduced pathogenicity compared to other viral vectors, episomal localization, and stable transgene expression. AAV viral particles show huge promise for the delivery of therapeutic genes to the liver. Improving the transduction efficiency of AAV particles having tropism for hepatic cells would be of great benefit to disease of the liver, including Alpha-1 Antitrypsin Deficiency and Transthyretin-Related Familial Amyloid Polyneuropathy. AAV virions of serotypes 3 and 3B have been demonstrated to possess tropism for liver cells. See Li et al., Mol Ther. 2015; 23(12): 1867-1876 and Glushakova, L G et al., Mol Genet Metab. 2009; 98: 289-299, each of which are herein incorporated by reference. This in part due to AAV3B's use of human hepatocyte growth factor receptor (huHGFR) as a cellular coreceptor.

The tissue tropism and transduction efficiency of AAV particles is determined by the nature of amino acid residues exposed at the surface of the capsid (Wu et al., J Virol. 2006, 80(22):11393-7, herein incorporated by reference). Therefore, manipulating the amino acids of the capsid proteins provides an opportunity to fine-tune the tissue tropism of the particle and also improve transduction efficiency. However, certain manipulations, e.g., substitutions of amino acids, of the capsid protein can cause it to mis-fold or not form a capsid at all. To circumvent issues of protein mis-folding and capsid mis-forming, the recombinant AAV3B (rAAV3B) variant proteins and viral particles disclosed herein were identified from a variant AAV3B capsid library that was built by making substitutions in only the variable loops of the capsid protein. Herein, “variable loops” are also referred to as “variable regions”. AAV3B has 9 variable regions, numbered from VRI to VRIX.

It was previously shown that pre-existing neutralized antibodies (NAb) against AAV3B are relatively lower (48% of animals with detectable NAb) as compared with AAV8 (≥75% of animals positive for AAV8 NAb) in non-human primates (see Li et al., Mol Ther. 2015). Screening of an AAV3B capsid library in a mouse model led to the identification of AAV3B capsid variants that possess enhanced efficiency to transduce hepatic cells compared to the transduction efficiency of wild-type AAV3B capsid proteins.

Accordingly, in some embodiments, the virions disclosed herein (e.g., E12 and G3 virions) may demonstrate reduced seroreactivity relative to a wild-type AAV3B capsid, or relative to another AAV3B variant capsid. In some embodiments, the virions disclosed herein may evade neutralizing antibodies (Nab) of host liver cells in vivo, e.g. in a subject, such as a primate (e.g., a human or a non-human primate). In some embodiments, the disclosed virions provide an about 1.5-fold, a 2-fold, a 2.5-fold, a 3-fold, a 3.2-fold, a 3.5-fold, a 4-fold, a 5-fold, a 6-fold, a 10-fold, a 12-fold or a 15-fold decrease in seroreactivity to neutralizing anti-AAV (e.g., anti-AAV3) antibodies in the subject, relative to a wild-type recombinant AAV3B virion. In some embodiments, the virions provide an about 2-fold decrease in seroreactivity to neutralizing anti-AAV antibodies in the subject, relative to a wild-type recombinant AAV3B virion.

Accordingly, provided herein are capsid mutants, or variants, of wild-type AAV3B, compositions of such particles and methods of using these compositions to transduce hepatic cells, exhibit reduced sero-reactivity, and/or evade a host humoral immune response.

Reduced seroreactivity and evasion of NAb in subjects may be measured by any method known in the art. In some embodiments, the degree of reduced seroreactivity and/or evasion of NAb is evaluated in vivo in human sera by measuring the differential expression of a protein encoded in the rAAV vector (which indicates the degree of transduction of that protein) of an administered virion in a sample obtained from a subject that had been administered the virions. In other embodiments, degree of reduced seroreactivity and/or evasion of NAb is evaluated in vitro by pre-incubating an rAAV virion encoding a protein with pooled IVIg, transducing one or more cells (e.g., human cells) with the pre-incubated virions, and measuring the differential percent of transduction (i.e., % expression of encoded protein) by flow cytometry between samples.

In some aspects, the present disclosure provides variants of the wild-type AAV3B capsid. The wild-type AAV3B capsid, VP1 region is set forth as SEQ ID NO: 1, below. In some embodiments, the variants, or modified capsids, of the present disclosure have an amino acid sequence essentially as set forth in SEQ ID NO: 1. In certain embodiments, the modified AAV capsid is truncated by 1, 2, 3, 4, 5, 6, 7, 8, 9, 10, 11, 12, 13, 14, 15, or 15-20 amino acids relative to the wild-type AAV3B VP1 sequence of SEQ ID NO: 1.

(SEQ ID NO: 1)
MAADGYLPDWLEDNLSEGIREWWALKPGVPQPKANQQHQDNRRGLVLPG
YKYLGPGNGLDKGEPVNEADAAALEHDKAYDQQLKAGDNPYLKYNHADA
EFQERLQEDTSFGGNLGRAVFQAKKRILEPLGLVEEAAKTAPGKKRPVD
QSPQEPDSSSGVGKSGKQPARKRLNFGQTGDSESVPDPQPLGEPPAAPT
SLGSNTMASGGGAPMADNNEGADGVGNSSGNWHCDSQWLGDRVITTSTR
TWALPTYNNHLYKQISSQSGASNDNHYFGYSTPWGYFDFNRFHCHFSPR
DWQRLINNNWGFRPKKLSFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFQFSYTFEDVPFHSSYAHSQSLDRLMNPLIDQY
LYYLNRTQGTTSGTTNQSRLLFSQAGPQSMSLQARNWLPGPCYRQQRLS
KTANDNNNSNFPWTAASKYHLNGRDSLVNPGPAMASHKDDEEKFFPMHG
NLIFGKEGTTASNAELDNVMITDEEEIRTTNPVATEQYGTVANNLQSSN
TAPTTRTVNDQGALPGMVWQDRDVYLQGPIWAKIPHTDGHFHPSPLMGG
FGLKHPPPQIMIKNTPVPANPPTTFSPAKFASFITQYSTGQVSVEIEWE
LQKENSKRWNPEIQYTSNYNKSVNVDFTVDTNGVYSEPRPIGTRYLTRN
L

In some embodiments, the modified AAV capsid comprises an amino acid sequence that is at least 80%, at least 85%, at least 90%, at least 95%, at least 98%, or at least 99.5% identical the sequence set forth as SEQ ID NO: 2. In some embodiments, the modified AAV capsid comprises 1, 2, 3, 4, 5, 6, 7, 8, 9, 10, 11, 12, 13, 14, 15, or 15-20 amino acid substitutions relative to the amino acid sequence of SEQ ID NO: 2. These differences may comprise amino acids that have been inserted, deleted, or substituted relative to the sequence of SEQ ID NO: 2. In some embodiments, the disclosed capsid rAAV variants comprise truncations at the N- or C-terminus relative to the sequence of SEQ ID NO: 2. In some embodiments, the disclosed capsid rAAV variants comprise stretches of 15, 20, 25, 30, 35, 40, 45, 50, or more than 50 consecutive amino acids in common with the sequence of SEQ ID NO: 2.

In some embodiments, the modified AAV capsid comprises the VP1 sequence of AAV3B-G3, or “G3,” which comprises the amino acid sequence set forth as SEQ ID NO: 2:

(SEQ ID NO: 2)
MAADGYLPDWLEDNLSEGIREWWALKPGVPQPKANQQHQDNRRGLVLPG
YKYLGPGNGLDKGEPVNEADAAALEHDKAYDQQLKAGDNPYLKYNHADA
EFQERLQEDTSFGGNLGRAVFQAKKRILEPLGLVEEAAKTAPGKKRPVD
QSPQEPDSSSGVGKSGKQPARKRLNFGQTGDSESVPDPQPLGEPPAAPT
SLGSNTMASGGGAPMADNNEGADGVGNSSGNWHCDSQWLGDRVITTSTR
TWALPTYNNHLYKQISSQSGASNDNHYFGYSTPWGYFDFNRFHCHFSPR
DWQRLINNNWGFRPKKLSFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFQFSYTFEDVPFHSSYAHSQSLDRLMNPLIDQY
LYYLNRTQSTASGTTGTSTLRFSQAGPQSMSLQARNWLPGPCYRQQRLS
KTANDNNNSNFPWTAASKYHLNGRDSLVNPGPAMASHKDDEEKFFPMHG
NLIFGKEGTTASNAELDNVMITDEEEIRTTNPVATEQYGTVANNLQNGR
DNPTFRDVQHQGALPGMVWQDRDVYLQGPIWAKIPHTDGHFHPSPLMGG
FGLKHPPPQIMIKNTPVPANPPTTFSPAKFASFITQYSTGQVSVEIEWE
LQKENSKRWNPEIQYTSNYNKSVNVDFTVDTNGVYSEPRPIGTRYLTRN
L

In some embodiments, the modified AAV capsid comprises an amino acid sequence that is at least 80%, at least 85%, at least 90%, at least 95%, at least 98%, or at least 99.5% identical the sequence set forth as SEQ ID NO: 10. In some embodiments, the modified AAV capsid comprises 1, 2, 3, 4, 5, 6, 7, 8, 9, 10, 11, 12, 13, 14, 15, or 15-20 amino acid substitutions relative to the amino acid sequence of SEQ ID NO: 10. These differences may comprise amino acids that have been inserted, deleted, or substituted relative to the sequence of SEQ ID NO: 10. In some embodiments, the disclosed capsid rAAV variants comprise truncations at the N- or C-terminus relative to the sequence of SEQ ID NO: 10. In some embodiments, the disclosed capsid rAAV variants comprise stretches of 15, 20, 25, 30, 35, 40, 45, 50, or more than 50 consecutive amino acids in common with the sequence of SEQ ID NO: 10.

In some embodiments, the modified AAV capsid comprises the VP1 sequence of AAV3B-E12, or “E12,” which comprises the amino acid sequence set forth as SEQ ID NO: 10:

(SEQ ID NO: 10)
MAADGYLPDWLEDNLSEGIREWWALKPGVPQPKANQQHQDNRRGLVLPG
YKYLGPGNGLDKGEPVNEADAAALEHDKAYDQQLKAGDNPYLKYNHADA
EFQERLQEDTSFGGNLGRAVFQAKKRILEPLGLVEEAAKTAPGKKRPVD
QSPQEPDSSSGVGKSGKQPARKRLNFGQTGXSESVPDPQPLGEPPAAPT
SLGSNTMASGGGAPMADNNEGADGVGNSSGNWHCDSQWLGDRVITTSTR
TWALPTYNNHLYKQISSQSGATNDNHYFGYSTPWGYFDFNRFHCHFSPR
DWQRLINNNWGFRPKKLSFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFQFSYTFEDVPFHSSYAHSQSLDRLMNPLIDQY
LYYLNRTQSTASGTTGTSTLRFSQAGPQSMSLQARNWLPGPCYRQQRLS
KIPGQNNNSNFPWTAASTYHLNGRDSLVNPGPAMASHKDDDERFFPMHG
NLIFGKQDTARSDVEVGKVMITDEEEIRTTNPVATEQYGTVANNLQSSN
TAPTTRTVNDQGALPGMVWQDRDVYLQGPIWAKIPHTDGHFHPSPLMGG
FGLKHPPPQIMIKNTPVPANPPTTFSPAKFASFITQYSTGQVSVEIEWE
LQKENSKRWNPEIQYTSNYNKSVNVDFTVDTNGVYSEPRPIGTRYLTRN
L

Accordingly, provided herein are rAAV3B capsid proteins comprising substitutions relative to the wild-type AAV3B VP1 sequence (e.g., as set forth in SEQ ID NO: 1). In some embodiments, an amino acid substitution in any one of the variant AAV3B capsid proteins disclosed herein lies in a variable region as defined by wild-type AAV3B VP1 protein. It should be understood that any positioning of an amino acid as described herein is with respect to the sequence of the wild-type AAV3B VP1 sequence as set forth in SEQ ID NO: 1.

In some embodiments, a variant rAAV3B capsid comprises one or more amino acid substitutions in any one variable region (e.g., VRI, VRII, VRIII, VRIV, VRV, VRVI, VRVII, VRVIII or VRIX). In some embodiments, a variant rAAV3B capsid comprises one or more amino acid substitutions in more than one variable region (e.g., VRI and VRII, VRI and VRVII or VRIV, VRII).

Certain aspects of the modified AAV capsids and AAV virions of the present disclosure include the second nucleotide encoding variants of an AAV Cap protein as listed in Table 4 (whose sequences are numbered 2-86 (SEQ ID NOs: 45-129)).

In some aspects, the present disclosure provides novel infectious rAAV virions and viral particles, as well as expression constructs that encode novel AAV virions. The present disclosure further provides novel nucleic molecules encoding one or more selected diagnostic and/or therapeutic agents for delivery to a selected population of mammalian cells, such as human cells, wherein the nucleic acid molecules are comprised within the disclosed rAAV virions and viral particles.

The present disclosure provides improved rAAV-based expression constructs that encode one or more therapeutic agents useful in the preparation of medicaments for the prevention, treatment, and/or amelioration of one or more diseases, disorders or conditions resulting from a deficiency in one or more cellular components. In particular, the present disclosure provides virions comprising modified capsids, as generated after screening of one or more libraries of rAAV-based genetic constructs encoding one or more selected molecules of interest, such as, for example, one or more diagnostic or therapeutic agents (including, e.g., proteins, polypeptides, peptides, antibodies, antigen binding fragments, siRNAs, RNAis, antisense oligo- and poly-nucleotides, ribozymes, and variants and/or active fragments thereof), for use in the diagnosis, prevention, treatment, and/or amelioration of symptoms of mammalian diseases, disorders, conditions, deficiencies, defects, trauma, injury, and such like.

In some embodiments, the novel capsids of the infectious virions disclosed herein may have an improved efficiency in transducing one or more of a variety of cells, tissues and organs of interest, when compared to wild-type, unmodified capsids. The improved rAAV capsids provided herein may transduce one or more selected host cells at higher-efficiencies (and often much higher efficiencies) than conventional, wild type (i.e., unmodified) rAAV capsids.

In the practice of the present disclosure, the transduction efficiency of a mutated rAAV capsid will be higher than that of the corresponding, unmodified, wild-type capsid, and as such, will preferably possess a transduction efficiency in a mammalian cell that is at least 2-fold, at least about 4-fold, at least about 6-fold, at least about 8-fold, at least about 10-fold, or at least about 12-fold or higher in a selected mammalian host cell than that of a virion that comprises a corresponding, unmodified rAAV capsid. In certain embodiments, the transduction efficiency of the rAAV capsids provided herein will be at least about 15-fold higher, at least about 20-fold higher, at least about 25-fold higher, at least about 30-fold higher, or at least about 40, 45, or 50-fold or more greater than that of a virion that comprises a corresponding, wild-type capsids.

The virions as described herein may be of different AAV serotypes, and the mutation of one or more of the sequences described herein may result in improved viral vectors, which are capable of higher-efficiency transduction than that of the corresponding, non-substituted vectors from which the mutants were prepared. In some embodiments, the virions as described herein may be of an AAV1, AAV2, AAV3, AAV4, AAV5, AAV6, AAV7, AAV8, AAV9, AAV10, AAV11, AAV12, or AAV13 serotype.

The present disclosure further provides populations and pluralities of the disclosed rAAV virions, infectious viral particles, and mammalian host cells that include one or more nucleic acid segments encoding them. The disclosed vectors and virions may be comprised within one or more diluents, buffers, physiological solutions or pharmaceutical vehicles, or formulated for administration to a mammal in one or more diagnostic, therapeutic, and/or prophylactic regimens. The disclosed viral particles, virions, and pluralities thereof may also be provided in excipient formulations that are acceptable for veterinary administration to selected livestock, exotics, domesticated animals, and companion animals (including pets and such like), as well as to non-human primates, zoological or otherwise captive specimens, and such like.

Preferably, the mammalian host cells will be human host cells, including, for example blood cells, stem cells, hematopoietic cells, CD34 cells, liver cells, cancer cells, vascular cells, pancreatic cells, neural cells, ocular or retinal cells, epithelial or endothelial cells, dendritic cells, fibroblasts, or any other cell of mammalian origin, including, without limitation, hepatic (i.e., liver) cells, lung cells, cardiac cells, pancreatic cells, intestinal cells, diaphragmatic cells, renal (i.e., kidney) cells, neural cells, blood cells, bone marrow cells, retinal cells or any one or more selected tissues of a mammal for which viral-based gene therapy is contemplated.

The present disclosure further provides compositions and formulations that include one or more of the host cells or viral particles of the present disclosure together with one or more pharmaceutically acceptable buffers, diluents, or carriers. Such compositions may be included in one or more diagnostic or therapeutic kits, for diagnosing, preventing, treating or ameliorating one or more symptoms of a mammalian disease, injury, disorder, trauma or condition.

The present disclosure further includes methods for providing a mammal in need thereof with a diagnostically- or therapeutically-effective amount of a selected biological molecule, the method comprising providing to a cell, tissue or organ of a mammal in need thereof, an amount of an rAAV expression construct; and for a time effective to provide the mammal with a diagnostically- or a therapeutically-effective amount of the selected biological molecule.

The present disclosure further provides methods for diagnosing, preventing, treating, or ameliorating at least one or more symptoms of a disease, a disorder, a condition, an injury, an abnormal condition, or trauma in a mammal. In an overall and general sense, the methods include at least the step of administering to a mammal in need thereof one or more of the disclosed rAAV constructs, in an amount and for a time sufficient to diagnose, prevent, treat or ameliorate the one or more symptoms of the disease, disorder, condition, injury, abnormal condition, or trauma in the mammal.

The present disclosure also provides methods of transducing a population of mammalian cells. In an overall and general sense, the methods include at least the step of introducing into one or more cells of the population, a composition that comprises an effective amount of one or more of the rAAV virions disclosed herein.

In other aspects, the present disclosure provides compositions, as well as therapeutic and/or diagnostic kits that include one or more of the disclosed AAV compositions, formulated with one or more additional ingredients, or prepared with one or more instructions for their use.

In some aspects, the present disclosure provides methods for using the disclosed improved rAAV virions in a variety of ways, including, for example, ex situ, ex vivo, in vitro and in vivo applications, methodologies, diagnostic procedures, and/or gene therapy regimens. Because many of the improved vectors described herein are also resistant to proteasomal degradation, they possess significantly increased transduction efficiencies in vivo making them particularly well suited for viral particle-based human gene therapy regimens, and in particular, for delivering one or more genetic constructs to selected mammalian cells in vivo and/or in vitro.

In one aspect, the present disclosure provides compositions comprising AAV virions, viral particles, and pharmaceutical formulations thereof, useful in methods for delivering genetic material encoding one or more beneficial or therapeutic product(s) to mammalian cells and tissues. In particular, the compositions and methods of the present disclosure provide a significant advancement in the art through their use in the treatment, prevention, and/or amelioration of symptoms of one or more mammalian diseases. It is contemplated that human gene therapy will particularly benefit from the present teachings by providing new and improved viral vector constructs for use in the treatment of a number of diverse diseases, disorders, and conditions.

Contemplated herein are also variant rAAV capsid proteins of serotypes other than serotype 3B. In some embodiments, any one of the amino acid substitutions described herein are in a variable region of the capsid protein of a serotype other than serotype 3B that is homologous to the variable region of AAV3B. In some embodiments, a variant rAAV capsid protein of a serotype other than serotype 3B is of any serotype other than AAV3B (e.g., 1, 2, 3, 4, 5, 6, 7, 8, 9, 10, 11, 12 or 13). In some embodiments, a variant rAAV capsid protein of a serotype other than serotype 3B is of a closely related serotype (e.g., AAV3).

### Library Design and Construction

In another aspect, the present disclosure concerns libraries of rAAV capsid variants that demonstrate improved properties useful in the delivery of one or more therapeutic agents to selected mammalian cells, and particularly for use in the prevention, treatment, and/or amelioration of one or more disorders in a mammal into which the vector construct may be introduced. In some embodiments, the disclosed libraries comprise rAAV3 capsid variants.

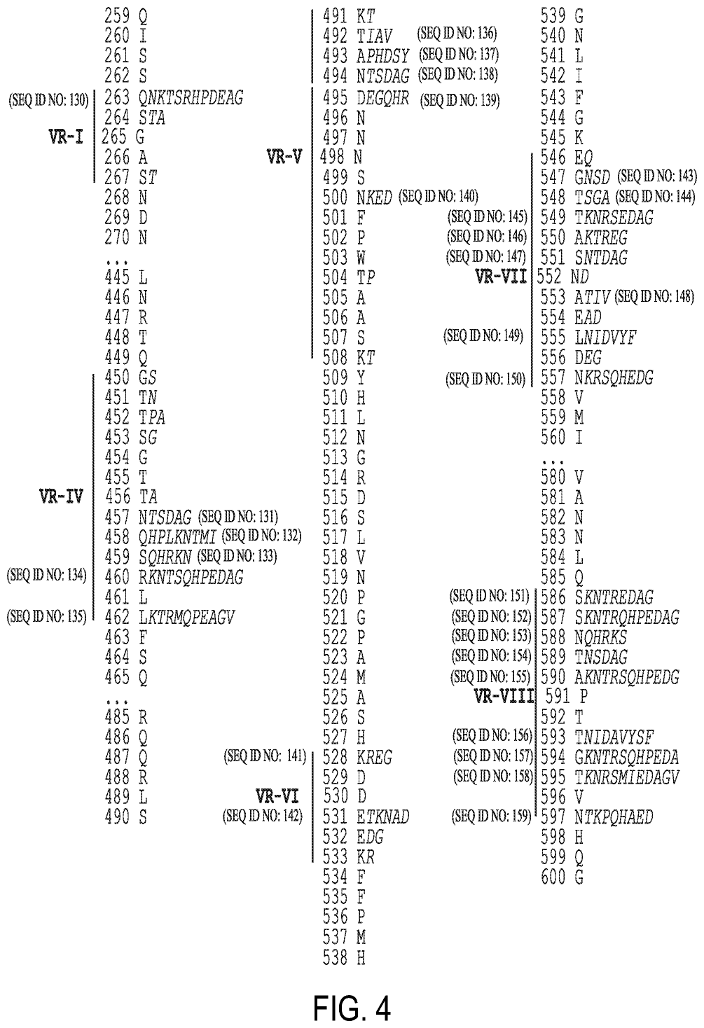

Comparison of the AAV VP3 structure among various serotypes has revealed highly homologous sequences interspersed with more evolutionary divergent areas. These amino acid stretches are commonly designated as VRs I through IX (variable regions I-IX; also known as “loops”). VRs are localized at the surface of the assembled capsid and are assumed to be responsible for the capsid interaction with cell surface receptors and other host factors. Because of their location, VRs are also predicted to be less critical for capsid assembly. Therefore, the guiding principle of the library's design was to modify only surface VRs while keeping the backbone sequence unchanged to maintain the integrity of the assembling scaffold. All candidate positions for mutagenesis in the AAV3 background, were selected from the alignment of known variants, which can be evaluated on a three dimensional model of the AAV3 capsid. The amino acid diversity of VR-I, VR-IV, VR-V, VR-VI, VR-VII and VR-VIII is shown in FIG. 4. AAV3 wildtype VR-II, VR-III and VR-IX and non-variable regions of VP3 were incorporated in the plasmid library. The wild-type AAV3B sequence is set forth in SEQ ID NO: 1.

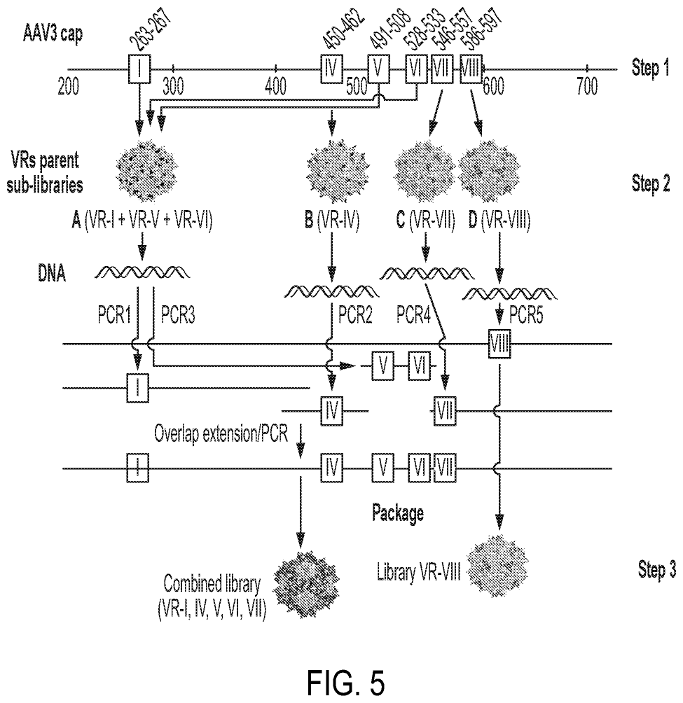

The AAV3B library of the present disclosure was built in three steps: first, VR parent sub-libraries were prepared each containing mutations in only one VR (B: VR-IV, C: VR-VII, D: VR-VIII) or a subset of VRs (A: VR-I+VR-V+VR-VI), then, structurally compatible sequences were combined to generate master libraries (A+B+C: VRs I, IV, V, VI, VII) and (D: VR-VIII), and finally the master libraries were packaged. See Example 1 and FIG. 5. Methods for generating and assembling DNA fragments for the library are disclosed in International Publication Nos. WO 2015/048534 and WO 2017/070476, and U.S. Pat. No. 7,220,577, each of which are incorporated herein by reference. The completed master library comprised 10⁷ variants.

The amino acid substitutions in the wild-type AAV3B capsid proteins disclosed herein are epistatic, i.e. that they interact with one another, e.g. synergistically. The disclosed substitutions may be grouped into motifs of substitutions. In designing the disclosed library, motifs were introduced to the capsids simultaneously and stochastically, rather than once at a time. The substitutions in each capsid variant were determined to be epistatic and act synergistically on capsid binding and transduction behavior. These motifs confer unexpectedly enhanced transduction efficiencies and may confer an ability to evade neutralizing antibodies relative to wild-type capsids of the prior art.

### Tissue-Specific or Cell-Specific Virions

The master library may be used to select virions having capsids containing degenerate or otherwise modified Cap protein (i.e., Cap protein that differs from wildtype serotype 3 at least at one amino acid position) that are targeted to particular tissue or cell types. For example, virions made according to the present disclosure include those that exhibit a new tropism, e.g., those capable of infecting cells normally non-permissive to AAV infection in general or at least non-permissive to AAV3 infection, as well as those that exhibit an increased or decreased ability to infect a particular cell or tissue type. As another example, virions made according to the present disclosure include those that lack the ability to infect cells normally permissive to AAV infection in general or at least normally permissive to AAV3 infection. To select for virions having a particular cell- or tissue-specific tropism, a packaged master library is introduced into a target cell. Preferably, the target cell is also infected with a helper virus (e.g. adenovirus, or Ad). The target cell is cultured under conditions that allow for the production of virions, resulting in a population of virions that are harvested from the target cell. This population of virions has been selected for having a tropism for that target cell.

As controls in a typical experiment in which virions having a particular tropism are selected, cells in different flasks or dishes may be simultaneously infected with WT AAV3 or rAAV using the same conditions as used for the library. After a suitable time post-infection, cells may be harvested, washed and the virions purified using a suitable purification method. See Gao et al., Hum. Gene Ther. 9:2353-62, 1998; U.S. Pat. No. 6,146,874; and Zolotukhin et al., Gene Ther. 6:973-85, 1999, each of which are incorporated herein by reference. AAV and helper virions (e.g., Ad) from each infection may be titered, e.g. by real-time PCR, and the AAV virions may then be further propagated, resulting in a stock of selected virions.

Once the selected population of virions having a desired tropism is isolated, nucleic acid from the virions is isolated and the sequence of the nucleotide sequence encoding the at least one AAV Cap protein is determined. Virions constructed and selected according to the present disclosure (e.g. virions comprising E12 and G3) that can specifically target diseased cells or tissues over non-diseased cells or tissues are particularly useful.

Alternatively, tissue- or cell-specific virions may be selected using an in vivo approach. For example, mice (or other suitable host) may be injected with a suitable amount of viral preparation (e.g., 1×10¹⁰ to 1×10¹¹ vector genomes (vg) in the case of mice) via the tail vein. As described in Example 2, more than one round of selection (iterative selection) may be performed by injecting the original master library for the first round and target-enriched libraries in subsequent rounds. Hosts are euthanized after an incubation period (3 to 4 days for mice), and episomal DNA is purified from the target cells or tissue and used as a template to amplify capsid DNA sequences. Target-enriched libraries may then be generated, purified and quantified. After several rounds of selection, amplified capsid DNA may be inserted into an appropriate vector for cloning and random clones may be analyzed by sequencing.

### Expression Constructs

In some aspects, the present disclosure provides polynucleotide expression constructs that encode one or more of the disclosed capsids as described herein. The expression construct may be comprised within a plasmid. These plasmids may comprise one or more nucleotide substitutions to the nucleic acid sequence that encodes a wild-type AAV3B capsid, e.g., one or more nucleotide substitutions in one or more variable regions.

In some embodiments, the nucleic acid vector comprises one or more transgenes comprising a sequence encoding a protein or polypeptide of interest operably linked to a promoter, wherein the one or more transgenes are flanked on each side with an ITR sequence. In some embodiments, the nucleic acid vector further comprises a region encoding a Rep protein as described herein, either contained within the region flanked by ITRs or outside the region or nucleic acid) operably linked to a promoter, wherein the one or more nucleic acid regions. The ITR sequences can be derived from any AAV serotype (e.g., 1, 2, 3, 4, 5, 6, 7, 8, 9, or 10) or can be derived from more than one serotype. In some embodiments, the ITR sequences are derived from AAV2 or AAV3. In other embodiments, the ITR sequences of the first serotype are derived from AAV1, AAV5, AAV6, AAV7, AAV8, AAV9 or AAV10. In some embodiments, the ITR sequences are the same serotype as the capsid (e.g., AAV3 ITR sequences and AAV3 capsid, etc.).

ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Ca; and Addgene, Cambridge, Mass.; and Gene delivery to skeletal muscle results in sustained expression and systemic delivery of a therapeutic protein. Kessler P D, et al. Proc Natl Acad Sci USA. 1996; 93(24):14082-7; and Curtis A. Machida, Methods in Molecular Medicine™ Viral Vectors for Gene Therapy Methods and Protocols. 10.1385/1-59259-304-6:201 Humana Press Inc. 2003: Chapter 10, Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In other aspects, the present disclosure provides rAAV nucleic acid vectors that may comprise a nucleic acid segment further comprises a promoter, an enhancer, a post-transcriptional regulatory sequence, a polyadenylation signal, or any combination thereof, operably linked to the nucleic acid segment that encodes he selected polynucleotide of interest. Preferably, the promoter is a heterologous promoter, a tissue-specific promoter, a cell-specific promoter, a constitutive promoter, an inducible promoter, or any combination thereof. Preferably, the expression constructs of the present disclosure further include at least promoter capable of expressing, or directed to primarily express, the nucleic acid segment in a suitable host cell (e.g., a liver cell) comprising the vector.

In certain embodiments, nucleic acid segments cloned into one or more of the novel rAAV nucleic acid vectors described herein will preferably express or encode one or more transgenes of interest. Such transgenes of interest may comprise polypeptides, peptides, ribozymes, peptide nucleic acids, siRNAs, RNAis, antisense oligonucleotides, antisense polynucleotides, antibodies, antigen binding fragments, growth factors, cytokines and other small therapeutic proteins, or any combination thereof. In certain embodiments, the one or more transgenes encode an antibody, secreted growth factor, or cytokine.

In some embodiments, the transgene of interest encodes a serine protease inhibitor. In certain embodiments, the transgene comprises the SERPINA1 gene (e.g., the human SERPINA1 gene), which encodes alpha-1-antitrypsin in humans (UniProtKB accession number: P01009). In some embodiments, the transgene encodes a transport protein. In certain embodiments, the transgene comprises the IIR gene (e.g., the human TTR gene), which encodes transthyretin in humans (UniProtKB accession number: P02766). In some embodiments, the transgene encodes a P-type ATPase. In certain embodiments, the transgene comprises the ATP7B gene (e.g., the human ATP7B gene), which encodes a copper-transporting P-type ATPase in humans (UniProtKB accession number: P35670). In some embodiments, the transgene encodes a carbamoyltransferase. In certain embodiments, the transgene comprises the OTC gene (e.g., the human OTC gene), which encodes ornithine transcarbamylase in humans (UniProtKB accession number: P00480).

Therapeutic agents useful in the disclosed vectors may include one or more agonists, antagonists, anti-apoptosis factors, inhibitors, receptors, cytokines, cytotoxins, erythropoietic agents, glycoproteins, growth factors, growth factor receptors, hormones, hormone receptors, interferons, interleukins, interleukin receptors, nerve growth factors, neuroactive peptides, neuroactive peptide receptors, proteases, protease inhibitors, protein decarboxylases, protein kinases, protein kinase inhibitors, enzymes, receptor binding proteins, transport proteins or one or more inhibitors thereof, serotonin receptors, or one or more uptake inhibitors thereof, serpins, serpin receptors, tumor suppressors, diagnostic molecules, chemotherapeutic agents, cytotoxins, or any combination thereof.

In exemplary embodiments, the rAAV nucleic acid vectors obtained by the disclosed methods may encode at least one diagnostic or therapeutic protein or polypeptide selected from the group consisting of a molecular marker, an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinsase inhibitor, a proteolytic protein, a proteolytic protein inhibitor, a semaphoring a semaphorin receptor, a serotonin transport protein, a serotonin uptake inhibitor, a serotonin receptor, a serpin, a serpin receptor, a tumor suppressor, and any combination thereof.

In certain applications, the rAAV nucleic acid vectors of the present disclosure may comprise one or more nucleic acid segments that encode a polypeptide selected from the group consisting of BDNF, CNTF, CSF, EGF, FGF, G-SCF, GM-CSF, gonadotropin, IFN, IFG-1, M-CSF, NGF, PDGF, PEDF, TGF, TGF-B2, TNF, VEGF, prolactin, somatotropin, XIAP1, IL-1, IL-2, IL-3, IL-4, IL-5, IL-6, IL-7, IL-8, IL-9, IL-10, IL-10(I87A), viral IL-10, IL-11, IL-12, IL-13, IL-14, IL-15, IL-16, IL-17, IL-18, and any combination thereof.

The rAAV nucleic acid vectors of the present disclosure may optionally further include one or more enhancer sequences that are each operably linked to the nucleic acid segment. Exemplary enhancer sequences include, but are not limited to, one or more selected from the group consisting of a CMV enhancer, a synthetic enhancer, a liver-specific enhancer, an vascular-specific enhancer, a brain-specific enhancer, a neural cell-specific enhancer, a lung-specific enhancer, a muscle-specific enhancer, a kidney-specific enhancer, a pancreas-specific enhancer, retinal-specific enhancer and an islet cell-specific enhancer.

Exemplary promoters useful in the practice of the present disclosure include, without limitation, one or more heterologous, tissue-specific, constitutive or inducible promoters, including, for example, but not limited to, a promoter selected from the group consisting of a CMV promoter, a β-actin promoter, an insulin promoter, an enolase promoter, a BDNF promoter, an NGF promoter, an EGF promoter, a growth factor promoter, an axon-specific promoter, a dendrite-specific promoter, a brain-specific promoter, a hippocampal-specific promoter, a kidney-specific promoter, a retinal-specific promoter, an elafin promoter, a cytokine promoter, an interferon promoter, a growth factor promoter, an α₁-antitrypsin promoter, a brain cell-specific promoter, a neural cell-specific promoter, a central nervous system cell-specific promoter, a peripheral nervous system cell-specific promoter, an interleukin promoter, a serpin promoter, a hybrid CMV promoter, a hybrid β-actin promoter, an EF1 promoter, a U1a promoter, a U1b promoter, a Tet-inducible promoter, a VP1 6-LexA promoter, or any combination thereof. In exemplary embodiments, the promoter may include a mammalian or avian β-actin promoter.

The vector-encoding nucleic acid segments may also further include one or more post-transcriptional regulatory sequences or one or more polyadenylation signals, including, for example, but not limited to, a woodchuck hepatitis virus post-transcription regulatory element (WPRE), a polyadenylation signal sequence, or any combination thereof.

In some aspects, the present disclosure concerns genetically-modified, improved-transduction-efficiency rAAV nucleic acid vectors that include at least a first nucleic acid segment that encodes one or more therapeutic agents that alter, inhibit, reduce, prevent, eliminate, or impair the activity of one or more endogenous biological processes in the cell. In particular embodiments, such therapeutic agents may be those that selectively inhibit or reduce the effects of one or more metabolic processes, conditions, disorders, or diseases. In certain embodiments, the defect may be caused by injury or trauma to the mammal for which treatment is desired. In other embodiments, the defect may be caused the over-expression of an endogenous biological compound, while in other embodiments still; the defect may be caused by the under-expression or even lack of one or more endogenous biological compounds.

The rAAV nucleic acid vectors of the present disclosure may also further optionally include a second distinct nucleic acid segment that comprises, consists essentially of, or consists of, one or more enhancers, one or more regulatory elements, one or more transcriptional elements, or any combination thereof, that alter, improve, regulate, and/or affect the transcription of the nucleotide sequence of interest expressed by the modified rAAV vectors.

For example, the rAAV nucleic acid vectors of the present disclosure may further include a second nucleic acid segment that comprises, consists essentially of, or consists of, a CMV enhancer, a synthetic enhancer, a cell-specific enhancer, a tissue-specific enhancer, or any combination thereof. The second nucleic acid segment may also further comprise, consist essentially of, or consist of, one or more intron sequences, one or more post-transcriptional regulatory elements, or any combination thereof.

The vectors of the present disclosure may also optionally further include a polynucleotide that comprises, consists essentially of, or consists of, one or more polylinkers, restriction sites, and/or multiple cloning region(s) to facilitate insertion (cloning) of one or more selected genetic elements, genes of interest, or therapeutic or diagnostic constructs into the rAAV construct at a selected site within the construct.

The disclosed nucleic acid vectors may be self-complementary (i.e., scrAAV nucleic acid vectors). In other embodiments, the vectors may be single-stranded.

The expression constructs and nucleic acid vectors of the present disclosure may be prepared in a variety of compositions, and may also be formulated in appropriate pharmaceutical vehicles for administration to human or animal subjects.

### Host Cells

The present disclosure also concerns host cells that comprise at least one or more of the disclosed virus particles or virions (e.g. virions comprising E12 and G3), or one or more of the disclosed rAAV expression constructs. Such host cells are particularly mammalian host cells, with human host cells being particularly preferred, and may be either isolated, in cell or tissue culture. In the case of genetically modified animal models, the transformed host cells may even be comprised within the body of a non-human animal itself. In some embodiments, the host cells comprise humanized host cells. In particular embodiments, the host cells comprise humanized hepatocytes.

Examples of suitable host cells include hepatocytes, such as H2.35, HUH-7 and HepG2, HEK293 embryonic kidney cells, HeLa cells, Cos cells, U87 cells, KB cells, and Vero cells. In certain embodiments, the modified AAV3B virions of the present disclosure are incorporated into HUH-7 and/or HepG2 cells. In particular embodiments, virions comprising the E12 and/or G3 variants are incorporated into HUH-7 and/or HepG2 cells.

As described above, the exogenous polynucleotide will preferably encode one or more proteins, polypeptides, peptides, ribozymes, or antisense oligonucleotides, or a combination of these. The exogenous polynucleotide may encode two or more such molecules, or a plurality of such molecules as may be desired. When combinational gene therapies are desired, two or more different molecules may be produced from a single rAAV expression construct, or alternatively, a selected host cell may be transfected with two or more unique rAAV expression constructs, each of which will provide unique transgenes encoding at least two different such molecules.

Use of rAAV Virions in Prophylaxis, Diagnosis, or Therapy

The present disclosure also provides for uses of the compositions disclosed herein as a medicament, or in the manufacture of a medicament, for treating, preventing or ameliorating the symptoms of a disease, disorder, condition, injury or trauma, including, but not limited to, the treatment, prevention, and/or prophylaxis of a disease, disorder or condition, and/or the amelioration of one or more symptoms of such a disease, disorder or condition.

In some embodiments, the disease, disorder or condition consists of Alpha-1 Antitrypsin Deficiency, Transthyretin-Related Familial Amyloid Polyneuropathy, Ornithine Transcarbamylase Deficiency, Fabry Disease, Pompe Disease, Galactosemia, Progressive Familial Intrahepatic Cholestasis Types 1, 2 and 3, Hereditary Angioedema, Hemophilia B, Hemophilia A, Phenylketonuria, Glycogen Storage Disease Type 1A, Wilson's Disease, or Citrullinemia.

In certain embodiments, the disease, disorder or condition is Alpha-1 Antitrypsin Deficiency, a rare inherited condition that results from deficiency of alpha-1 antitrypsin (AAT), a protein produced in the liver encoded by the SERPINA1 gene. AAT deficiency often leads to cirrhosis and other severe liver diseases, as well as emphysema and COPD in the lungs.

In certain embodiments, the disease, disorder or condition is Transthyretin-Related Familial Amyloid Polyneuropathy (FAP), or Familial Transthyretin Amyloidosis (FTA). FAP is a rare inherited condition that results from an abnormal accumulation of amyloid in the body's tissues, and in particular liver tissue, due to abnormal misfolding and aggregation of transthyretin. FAP is an autosomal dominant condition resulting from a mutation in the TTR gene. In the absence of a liver transplant, FAP is invariably fatal.

In certain embodiments, the disease, disorder or condition is Wilson's Disease. Wilson's disease is a rare inherited disorder that causes copper to accumulate in organs such as the liver. It is an autosomal recessive condition due to a mutation in the ATP7B gene, which encodes a P-type ATPase that transports copper into bile and incorporates it into ceruloplasmin.

In certain embodiments, the disease, disorder or condition is Ornithine Transcarbamylase Deficiency (OTC Deficiency), an inherited condition that results from a toxic accumulation of ammonia in the blood. It is an X-linked recessive condition due to a mutation in the OTC gene, which encodes a carbamoyltransferase that is expressed only in the liver and is responsible for converting carbamoyl phosphate and ornithine into citrulline as part of the urea cycle.

In certain embodiments, the creation of recombinant non-human host cells, humanized host cells, and/or isolated recombinant human host cells that comprise one or more of the disclosed rAAV virions (e.g. virions comprising E12 and/or G3) is also contemplated to be useful for a variety of diagnostic, and laboratory protocols, including, for example, means for the production of large-scale quantities of the virions described herein. Such virus production methods may comprise improvements over existing methodologies including in particular, those that require very high titers of the viral stocks in order to be useful as a gene therapy tool. The inventors contemplate that one very significant advantage of the present methods will be the ability to utilize lower titers of viral particles in mammalian transduction protocols, yet still retain transfection rates at a suitable level.

The present disclosure provides methods of transducing a hepatic cell with a transgene of interest, the method comprising providing to the hepatic cell any of the variant recombinant AAV particles of the disclosure. In some embodiments, the hepatic cell is a human hepatocyte.

Additional aspects of the present disclosure concern methods of use of the disclosed virions, expression constructs, compositions, and host cells in the preparation of medicaments for diagnosing, preventing, treating or ameliorating at least one or more symptoms of a disease, a condition, a disorder, an abnormal condition, a deficiency, injury, or trauma in an animal, and in particular, in a vertebrate mammal, e.g., Alpha-1 Antitrypsin Deficiency, Transthyretin-Related Familial Amyloid Polyneuropathy, Ornithine Transcarbamylase Deficiency, or Wilson's Disease. Such methods generally involve administration to a mammal in need thereof, one or more of the disclosed virions, host cells, compositions, or pluralities thereof, in an amount and for a time sufficient to diagnose, prevent, treat, or lessen one or more symptoms of such a disease, condition, disorder, abnormal condition, deficiency, injury, or trauma in the affected animal. The methods may also encompass prophylactic treatment of animals suspected of having such conditions, or administration of such compositions to those animals at risk for developing such conditions either following diagnosis, or prior to the onset of symptoms.

The present disclosure also provides a method for treating or ameliorating the symptoms of such a disease, injury, disorder, or condition in a mammal. Such methods generally involve at least the step of administering to a mammal in need thereof, one or more of the rAAV virions as disclosed herein, in an amount and for a time sufficient to treat or ameliorate the symptoms of such a disease, injury, disorder, or condition in the mammal. Such treatment regimens are particularly contemplated in human therapy, via administration of one or more compositions either intramuscularly, intravenously, subcutaneously, intrathecally, intraperitoneally, or by direct injection into an organ or a tissue of the mammal under care.

The present disclosure also provides a method for providing to a mammal in need thereof, a therapeutically-effective amount of an rAAV composition of the present disclosure, in an amount, and for a time effective to provide the patient with a therapeutically-effective amount of the desired therapeutic agent(s) encoded by one or more nucleic acid segments comprised within the rAAV virion, e.g. a virion comprising E12 and/or G3. Exemplary therapeutic agents include, but are not limited to, a polypeptide, a peptide, an antibody, an antigen-binding fragment, a cytokine, a ribozyme, a peptide nucleic acid, an siRNA, an RNAi, an antisense oligonucleotide, an antisense polynucleotide, or a combination thereof.

Because the rAAV capsid variants of the disclosure possess enhanced ability to reduce seroreactivity and evade neutralizing antibodies, the compositions and methods provided herein facilitate the re-dosing or re-administration of an rAAV particle comprising any of the disclosed capsid variants to a subject who has been administered an rAAV particle previously, e.g., as part of a therapeutic regimen. This reduced seroreactivity likewise facilitates the first administration of an rAAV particle to a subject who had exposure to rAAVs previously naively, or outside of the context of a therapeutic regimen. In some embodiments, these subject are human.

Accordingly, the present disclosure provides re-dosing regimens of rAAV. In some aspects of the disclosure, methods of re-administration of rAAV particles (or virions) are provided. Such methods may comprise a first administration, followed by a subsequent (or second) administration of an rAAV particle comprising any of the disclosed capsid variants. In some embodiments, such methods comprise re-administering the recombinant AAV particle or a composition comprising such a particle to the subject, e.g., a human subject in need thereof whom has previously been administered the recombinant AAV particle or the composition.

### Pharmaceutical Compositions and Kits

In further aspects, the present disclosure provides compositions comprising one or more of the disclosed rAAV virions (e.g. virions comprising E12 and/or G3), expression constructs, infectious AAV particles, or host cells. In some embodiments, provided herein are compositions of rAAV virions that further comprise a pharmaceutically acceptable carrier for use in therapy, and for use in the manufacture of medicaments for the treatment of one or more mammalian diseases, disorders, conditions, or trauma (e.g., AAT or FAP). Such pharmaceutical compositions may optionally further comprise one or more diluents, buffers, liposomes, a lipid, a lipid complex, a microsphere or a nanoparticle.

In some embodiments, the disclosure provides pharmaceutical compositions that comprise a modified rAAV vector as disclosed herein, and further comprise a pharmaceutical excipient, and may be formulated for administration to host cell ex vivo or in situ in an animal, and particularly a human. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Such compositions may be formulated for use in a variety of therapies, such as for example, in the amelioration, prevention, and/or treatment of conditions such as peptide deficiency, polypeptide deficiency, peptide overexpression, polypeptide overexpression, including for example, conditions, diseases or disorders as described herein.

In some embodiments, the number of rAAV particles administered to a subject may be on the order ranging from 10⁶ to 10¹⁴ particles/mL or 10³ to 10¹³ particles/mL, or any values therebetween for either range, such as for example, about 10⁶, 10⁷, 10⁸, 10⁹, 10¹⁰, 10¹¹, 10¹², 10¹³, or 10¹⁴ particles/mL. In one embodiment, rAAV particles of higher than 10¹³ particles/mL are be administered. In some embodiments, the number of rAAV particles administered to a subject may be on the order ranging from 10⁶ to 10¹⁴ vector genomes (vgs)/mL or 10³ to 10¹⁵ vgs/mL, or any values there between for either range, such as for example, about 10⁶, 10⁷, 10⁸, 10⁹, 10¹⁰, 10¹¹, 10¹², 10¹³, or 10¹⁴ vgs/mL. In some embodiments, a dose of between 1×10¹² and 4×10¹² vgs/ml (or between 5×10¹¹ to 2×10¹² vgs/kg of the subject) is administered to the subject.

The rAAV particles can be administered as a single dose, or divided into two or more administrations as may be required to achieve therapy of the particular disease or disorder being treated.

In some embodiments, where a second nucleic acid vector encoding the Rep protein within a second rAAV particle is administered to a subject, the ratio of the first rAAV particle to the second rAAV particle is 1:100, 1:50, 1:40, 1:30, 1:20, 1:10, 1:5, 1:2 or 1:1. In some embodiments, the Rep protein is delivered to a subject such that target cells within the subject receive at least two Rep proteins per cell.

In some embodiments, the disclosure provides formulations of compositions disclosed herein in pharmaceutically acceptable carriers for administration to a cell or an animal, either alone or in combination with one or more other modalities of therapy, and in particular, for therapy of human cells, tissues, and diseases affecting man.

If desired, rAAV particle or preparation, Rep proteins, and nucleic acid vectors may be administered in combination with other agents as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The rAAV particles or preparations, Rep proteins, and nucleic acid vectors may thus be delivered along with various other agents as required in the particular instance. Such compositions may be purified from host cells or other biological sources, or alternatively may be chemically synthesized as described herein.

The formulation of pharmaceutically acceptable carriers is well-known to those of skill in the art, as is the development of suitable dosing and treatment regimens for using the particular compositions described herein in a variety of treatment regimens, including e.g., oral, parenteral, intravenous, intranasal, intra-articular, and intramuscular administration and formulation.

Typically, these formulations may contain at least about 0.1% of the therapeutic agent (e.g., rAAV particle or preparation, Rep protein, and/or nucleic acid vector) or more, although the percentage of the active ingredient(s) may, of course, be varied and may conveniently be between about 1% or 2% and about 70% or 80% or more of the weight or volume of the total formulation. Naturally, the amount of therapeutic agent(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV particles or preparations, Rep proteins, and/or nucleic acid vectors in suitably formulated pharmaceutical compositions disclosed herein either subcutaneously, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection.

The pharmaceutical forms of the compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

The pharmaceutical compositions of the present disclosure can be administered to the subject being treated by standard routes including, but not limited to, pulmonary, intranasal, oral, inhalation, parenteral such as intravenous, topical, transdermal, intradermal, transmucosal, intraperitoneal, intramuscular, intracapsular, intraorbital, intravitreal, intracardiac, transtracheal, subcutaneous, subcuticular, intraarticular, subcapsular, subarachnoid, intraspinal, epidural and intrasternal injection.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subcutaneous and intraperitoneal administration. To this end, a sterile aqueous medium that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Ed., 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions are prepared by incorporating the rAAV particles or preparations, Rep proteins, and/or nucleic acid vectors, in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Ex vivo delivery of cells transduced with rAAV particles or preparations, and/or Rep proteins is also contemplated herein. Ex vivo gene delivery may be used to transplant rAAV-transduced host cells back into the host. A suitable ex vivo protocol may include several steps. For example, a segment of target tissue or an aliquot of target fluid may be harvested from the host and rAAV particles or preparations, and/or Rep proteins may be used to transduce a nucleic acid vector into the host cells in the tissue or fluid. These genetically modified cells may then be transplanted back into the host. Several approaches may be used for the reintroduction of cells into the host, including intravenous injection, intraperitoneal injection, or in situ injection into target tissue. Autologous and allogeneic cell transplantation may be used according to the invention.

The amount of rAAV particle or preparation, Rep protein, or nucleic acid vector compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the rAAV particle or preparation, Rep protein, or nucleic acid vector compositions, either over a relatively short, or a relatively prolonged period of time, as may be determined by the medical practitioner overseeing the administration of such compositions.

Toxicity and efficacy of the compositions utilized in methods of the disclosure can be determined by standard pharmaceutical procedures, using either cells in culture or experimental animals to determine the LD₅₀ (the dose lethal to 50% of the population). The dose ratio between toxicity and efficacy the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of compositions as described herein lies generally within a range that includes an ED₅₀ with little or no toxicity. The dosage may vary within this range depending upon the dosage form employed and the route of administration utilized.

The present disclosure provides compositions including one or more of the disclosed rAAV virions (e.g., virions comprising E12 and/or G3) comprised within a kit for diagnosing, preventing, treating or ameliorating one or more symptoms of a mammalian disease, injury, disorder, trauma or condition. In some embodiments, the disease or disorder is AAT or FAP. Such kits may also be useful in the diagnosis, prophylaxis, and/or therapy or a human disease, and may be particularly useful in the treatment, prevention, and/or amelioration of one or more symptoms of Alpha-1 Antitrypsin Deficiency, Transthyretin-Related Familial Amyloid Polyneuropathy, Ornithine Transcarbamylase Deficiency, Wilson's Disease, wet age-related macular degeneration, dry age-related macular degeneration, glaucoma, retinitis pigmentosa, diabetic retinopathy, orphan ophthalmological diseases, cancer, diabetes, autoimmune disease, kidney disease, cardiovascular disease, pancreatic disease, intestinal disease, liver disease, neurological disease, neuromuscular disorder, neuromotor deficit, neuroskeletal impairment, neurological disability, neurosensory condition, stroke, ischemia, Batten's disease, Alzheimer's disease, sickle cell disease, β-thalassemia, Huntington's disease, Parkinson's disease, skeletal disease, trauma, pulmonary disease in a human.

Kits comprising one or more of the disclosed rAAV virions, transformed host cells or pharmaceutical compositions comprising such vectors; and instructions for using such kits in one or more therapeutic, diagnostic, and/or prophylactic clinical embodiments are also provided in the present disclosure. Such kits may further comprise one or more reagents, restriction enzymes, peptides, therapeutics, pharmaceutical compounds, or means for delivery of the composition(s) to host cells, or to an animal (e.g., syringes, injectables, and the like). Exemplary kits include those for treating, preventing, or ameliorating the symptoms of a disease, deficiency, condition, and/or injury, or may include components for the large-scale production of the viral vectors themselves, such as for commercial sale, or for use by others, including e.g., virologists, medical professionals, and the like.

Methods of Making rAAV3B Particles

Various methods of producing rAAV particles (e.g. particles comprising E12 and/or G3) and nucleic acid vectors are known (see, e.g., Zolotukhin et al. Methods 28 (2002) 158-167; and U.S. Patent Publication Nos. US 2007/0015238 and US 2012/0322861, each of which are incorporated herein by reference; and plasmids and kits available from ATCC and Cell Biolabs, Inc.). In some embodiments, a vector (e.g., a plasmid) comprising a transgene of interest may be combined with one or more helper plasmids, e.g., that contain a rep gene (e.g., encoding Rep78, Rep68, Rep52 and Rep40) and a cap gene (encoding VP1, VP2, and VP3, including a modified VP region as described herein), and transfected into a recombinant cells, called helper or producer cells, such that the nucleic acid vector is packaged or encapsidated inside the capsid and subsequently purified.

Non-limiting examples of mammalian helper cells include HEK293 cells, COS cells, HeLa cells, BHK cells, or CHO cells (see, e.g., ATCC® CRL-1573™, ATCC® CRL-1651™, ATCC® CRL-1650™, ATCC® CCL-2, ATCC® CCL-10™, or ATCC® CCL-61™). A non-limiting example of an insect helper cells is Sf9 cells (see, e.g., ATCC® CRL-1711™). A helper cell may comprises rep and/or cap genes that encode the Rep protein and/or Cap proteins. In some embodiments, the packaging is performed in vitro (e.g., outside of a cell).

In some embodiments, a nucleic acid vector (e.g., a plasmid) containing the transgene of interest (e.g., SERPINA1, TTR, ATP7B or OTC) is combined with one or more helper plasmids, e.g., that contain a rep gene of a first serotype and a cap gene of the same serotype or a different serotype, and transfected into helper cells such that the rAAV particle is packaged. In some embodiments, the one or more helper plasmids include a first helper plasmid comprising a rep gene and a cap gene, and a second helper plasmid comprising one or more of the following helper genes: Ela gene, E1b gene, E4 gene, E2a gene, and VA gene. For clarity, helper genes are genes that encode helper proteins Ela, E1b, E4, E2a, and VA. Helper plasmids, and methods of making such plasmids, are known in the art and commercially available (see, e.g., pDF6, pRep, pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Ca; and Addgene, Cambridge, Mass.; pxx6; Grimm et al. (1998), Novel Tools for Production and Purification of Recombinant Adeno associated Virus Vectors, Human Gene Therapy, Vol. 9, 2745-2760; Kern, A. et al. (2003), Identification of a Heparin-Binding Motif on Adeno-Associated Virus Type 2 Capsids, J. Virol., Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188). Plasmids that encode wild-type AAV coding regions for specific serotypes are also know and available. For example pSub201 is a plasmid that comprises the coding regions of the wild-type AAV2 genome (Samulski et al. (1987), J Virology, 6:3096-3101).

Inverted terminal repeat (ITR) sequences and plasmids containing ITR sequences are known in the art and are commercially available (see, e.g., products and services available from Vector Biolabs, Philadelphia, Pa.; Cellbiolabs, San Diego, Calif.; Agilent Technologies, Santa Clara, Ca; and Addgene, Cambridge, Mass.; and Gene delivery to skeletal muscle results in sustained expression and systemic delivery of a therapeutic protein. Kessler P D, et al. Proc Natl Acad Sci USA. 1996 November; 93(24):14082-7; and Curtis A. Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman, et al.; U.S. Pat. Nos. 5,139,941 and 5,962,313, all of which are incorporated herein by reference).

Genbank reference numbers for sequences of AAV serotype 3B are listed in patent publication WO 2012/064960, which is incorporated herein by reference in its entirety.

A non-limiting method of rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. In some embodiments, the one or more helper plasmids comprise rep genes, cap genes, and optionally one or more of the adenoviral VA, E2A (DBP), and E4 genes under the transcriptional control of their native promoters. In some embodiments, the one or more helper plasmids comprise cap ORFs (and optionally rep ORFs) for the desired AAV serotype and the adenoviral VA, E2A (DBP), and E4 genes under the transcriptional control of their native promoters. The cap ORF may also comprise one or more modifications to produce a modified capsid protein as described herein. As an example, HEK293 cells (available from ATCC®) are transfected via CaPO₄-mediated transfection, lipids or polymeric molecules such as Polyethylenimine (PEI) with the helper plasmid(s) and a plasmid containing a nucleic acid vector. The HEK293 cells are then incubated for at least 60 hours to allow for rAAV particle production. Alternatively, the HEK293 cells are transfected via methods described above with AAV-ITR containing one or more genes of interest, a helper plasmid comprising genes encoding Rep and Cap proteins, and co-infected with a helper virus. Helper viruses are viruses that allow the replication of AAV. Examples of helper virus are adenovirus (e.g., Ad5) and herpesvirus.

Alternatively, in another example, Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known in the art or described herein, e.g., by iodixanol step gradient, CsCl gradient, chromatography, or polyethylene glycol (PEG) precipitation. See US Patent Publication No. 2017/0130208, incorporated herein by reference.

Methods for large-scale production of AAV using a herpesvirus-based system are also known. See for example, Clement et al., Hum Gene Ther. 2009, 20(8):796-806. Methods of producing exosome-associated AAV, which can be more resistant to neutralizing anti-AAV antibodies, are also known (Hudry et al., Gene Ther. 2016, 23(4):380-92; Macguire et al., Mol Ther. 2012, 20(5):960-71).

Methods for producing and using pseudotyped rAAV vectors are also known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Illustrative embodiments of the present disclosure are described below. In the interest of clarity, not all features of an actual implementation are described in this specification. It will be appreciated by one of skill in the art that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

### Exemplary Definitions

Unless otherwise defined, all technical terms used herein have the same meaning as commonly understood by one of ordinary skill in the art to which this present disclosure belongs. Commonly understood definitions of molecular biology terms can be found in Rieger et al., (1991) and Lewin (1994). Commonly understood definitions of virology terms can be found in Granoff and Webster (1999) and Tidona and Darai (2002).

In accordance with convention, the words “a” and “an” when used in this application, including the claims, denotes “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

As used herein, the term “carrier” is intended to include any solvent(s), dispersion medium, coating(s), diluent(s), buffer(s), isotonic agent(s), solution(s), suspension(s), colloid(s), inert(s) or such like, or a combination thereof, that is pharmaceutically acceptable for administration to the relevant animal. The use of one or more delivery vehicles for chemical compounds in general, and chemotherapeutics in particular, is well known to those of ordinary skill in the pharmaceutical arts. Except insofar as any conventional media or agent is incompatible with the active ingredient, its use in the diagnostic, prophylactic, and therapeutic compositions is contemplated. One or more supplementary active ingredient(s) may also be incorporated into, or administered in association with, one or more of the disclosed chemotherapeutic compositions.

The term “e.g.,” as used herein, is used merely by way of example, without limitation intended, and should not be construed as referring only those items explicitly enumerated in the specification.

As used herein, “an effective amount” would be understood by those of ordinary skill in the art to provide a therapeutic, prophylactic, or otherwise beneficial effect against the organism, its infection, or the symptoms of the organism or its infection, or any combination thereof.

The phrase “expression control sequence” refers to any genetic element (e.g., polynucleotide sequence) that can exert a regulatory effect on the replication or expression (transcription or translation) of another genetic element. Common expression control sequences include promoters, polyadenylation (polyA) signals, transcription termination sequences, upstream regulatory domains, origins of replication, internal ribosome entry sites (IRES), enhancers, and the like. A “tissue specific expression control sequence” is one that exerts a regulatory effect on the replication or expression (transcription or translation) of another genetic element in only one type of tissue or a small subset of tissues.

The phrase “helper function” is meant as a functional activity performed by a nucleic acid or polypeptide that is derived from a virus such as Adenovirus (Ad) or herpesvirus and that facilitates AAV replication in a host cell.

As used herein, a “heterologous” is defined in relation to a predetermined referenced gene sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

As used herein, the term “homology” refers to a degree of complementarity between two or more polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity may be determined by analyzing two or more sequences using algorithms and computer programs known in the art. Such methods may be used to assess whether a given sequence is identical or homologous to another selected sequence.

As used herein, “homologous” means, when referring to polynucleotides, sequences that have the same essential nucleotide sequence, despite arising from different origins. Typically, homologous nucleic acid sequences are derived from closely related genes or organisms possessing one or more substantially similar genomic sequences. By contrast, an “analogous” polynucleotide is one that shares the same function with a polynucleotide from a different species or organism, but may have a significantly different primary nucleotide sequence that encodes one or more proteins or polypeptides that accomplish similar functions or possess similar biological activity. Analogous polynucleotides may often be derived from two or more organisms that are not closely related (e.g., either genetically or phylogenetic ally).

As used herein, the terms “humanize” and “humanized” refers to the action of engrafting human cells or tissues into a non-human animal, such as a mouse. The present disclosure may refer to humanized murine models and/or subjects, such as mouse models humanized with primary human hepatic cells.

The terms “identical” or percent “identity” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of ordinary skill) or by visual inspection.

As used herein, the term “in need of treatment” refers to a judgment made by a caregiver such as a physician or veterinarian that a patient requires (or will benefit in one or more ways) from treatment. Such judgment may made based on a variety of factors that are in the realm of a caregiver's expertise, and may include the knowledge that the patient is ill as the result of a disease state that is treatable by one or more compound or pharmaceutical compositions such as those set forth herein.

The terms “isolated” or “biologically pure” refer to material that is substantially, or essentially, free from components that normally accompany the material as it is found in its native state. Thus, isolated polynucleotides in accordance with the present disclosure preferably do not contain materials normally associated with those polynucleotides in their natural, or in situ, environment.

As used herein, the term “kit” may be used to describe variations of the portable, self-contained enclosure that includes at least one set of components to conduct one or more of the diagnostic or therapeutic methods of the present disclosure.

“Link” or “join” refers to any method known in the art for functionally connecting one or more proteins, peptides, nucleic acids, or polynucleotides, including, without limitation, recombinant fusion, covalent bonding, disulfide bonding, ionic bonding, hydrogen bonding, electrostatic bonding, and the like.

The term “library” refers to a collection of elements that differ from one another in at least one aspect. For example, a vector library is a collection of at least two vectors that differ from one another by at least one nucleotide. As another example, a “virion library” is a collection of at least two virions that differ from one another by at least one nucleotide or at least one capsid protein.

As used herein, the term “master library” or “combined library” refers to a pool of rAAV virions composed of chimeric rcAAV nucleic acid vectors encapsidated in cognate chimeric capsids (e.g., capsids containing a degenerate or otherwise modified Cap protein). As used herein, the term “rcAAV nucleic acid vector” refers to a replication-competent AAV-derived nucleic acid capable of DNA replication in a cell without any additional AAV genes or gene products.

As used herein, the term “parent sub-library” refers to a pool of rAAV virions composed of chimeric rcAAV nucleic acid vectors encapsidated in cognate chimeric capsids (e.g., capsids containing degenerate or otherwise modified Cap protein). More than one parent sub-library may be combined to create a master library or combined library.

When referring to a nucleic acid molecule or polypeptide, the term “native” refers to a naturally-occurring (e.g., a WT) nucleic acid or polypeptide.

The terms “naturally-occurring” or “native,” as used herein refers to the fact that the described molecule may be found in nature. For example, a polypeptide or polynucleotide sequence that is present in an organism (including viruses) that may be isolated from a source in nature and which has not been intentionally modified by the hand of man in a laboratory is naturally-occurring. As used herein, laboratory strains of rodents that may have been selectively bred according to classical genetics are considered naturally occurring animals.

As used herein, the phrase “nucleic acid” means a chain of two or more nucleotides such as RNA (ribonucleic acid) and DNA (deoxyribonucleic acid). Conventional nomenclature exists in the art for polynucleotide and polypeptide structures. For example, one-letter abbreviations are widely employed to describe nucleotides: Adenine (A), Guanine (G), Cytosine (C), Thymine (T), Uracil (U), Purine, i.e. A or G (R), Pyrimidine, i.e. C or T (Y), any nucleotide (N), Weak, i.e. A or T (W), Strong, i.e. G or C (S), Amino, i.e. A or C (M), Keto, i.e. G or T (K), not A, i.e. G or C or T (B), not G, i.e. A or C or T (H), not C, i.e. A or G or T (D) and not T, i.e. A or G or C (V).

In accordance with the present disclosure, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

The phrases “cap nucleic acid,” “cap gene,” and “capsid gene” as used herein mean a nucleic acid that encodes a Cap protein. Examples of cap nucleic acids include “wild-type” (WT) Cap-encoding nucleic acid sequences from AAV serotypes 1, 2, 3, 4, 5, 6, 7, 8, 9, 10, 11, 12, 13; a native form cap cDNA; a nucleic acid having sequences from which a cap cDNA can be transcribed; and/or allelic variants and homologs of the foregoing.

“VR”, “VRs”, “variable region” or “variable regions” refer to amino acid stretches of capsid protein that do not have a high degree of homology between AAV variants. These amino acid stretches are commonly designated as VRs I through IX (also known as “loops”). VRs are localized at the surface of the assembled capsid and interact with host cell surface receptors and other host factors.

The phrase “pharmaceutically acceptable” refers to molecular entities and compositions that preferably do not produce an allergic or similar untoward reaction when administered to a mammal, and in particular, when administered to a human. As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof.

As used herein, the term “plasmid” or “vector” refers to a genetic construct that is composed of genetic material (i.e., nucleic acids). Typically, a plasmid or a vector contains an origin of replication that is functional in bacterial host cells, e.g., Escherichia coli, and selectable markers for detecting bacterial host cells including the plasmid. Plasmids and vectors of the present disclosure may include one or more genetic elements as described herein arranged such that an inserted coding sequence can be transcribed and translated in a suitable expression cells. In addition, the plasmid or vector may include one or more nucleic acid segments, genes, promoters, enhancers, activators, multiple cloning regions, or any combination thereof, including segments that are obtained from or derived from one or more natural and/or artificial sources.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures. For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Additional conventions include: Asn or Asp (B; Asx), Gln or Glu (Z; Glx), Leu or Ile (J; Xle), Selenocysteine (U; Sec), Pyrrolysine (O; Pyl) and Unknown (X; Unk). Amino acid residues described herein are preferred to be in the “L” isomeric form. However, residues in the “D” isomeric form may be substituted for any L-amino acid residue provided the desired properties of the polypeptide are retained.

The term “promoter,” as used herein refers to a region or regions of a nucleic acid sequence that regulates transcription.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from two to about 20 amino acid residues in length, oligopeptides from about 10 to about 100 amino acid residues in length, and longer polypeptides including those of about 100 or more amino acid residues in length. Furthermore, the term is also intended to include enzymes, i.e., functional biomolecules including at least one amino acid polymer. Polypeptides and proteins of the present disclosure also include polypeptides and proteins that are or have been post-translationally modified, and include any sugar or other derivative(s) or conjugate(s) added to the backbone amino acid chain.

The term “pseudotyped” is meant a nucleic acid or genome derived from a first AAV serotype that is encapsidated (packaged) into an AAV capsid containing at least one AAV Cap protein of a second serotype differing from the first serotype.

The term “recombinant” indicates that the material (e.g., a polynucleotide or a polypeptide) has been artificially or synthetically (non-naturally) altered by human intervention. The alteration may be performed on the material within or removed from, its natural environment or state. Specifically, e.g., a promoter sequence is “recombinant” when it is produced by the expression of a nucleic acid segment engineered by the hand of man. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid; and a “recombinant virus,” e.g., a recombinant AAV virus, is produced by the expression of a recombinant nucleic acid.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The terms “substantially corresponds to,” “substantially homologous,” or “substantial identity,” as used herein, denote a characteristic of a nucleic acid or an amino acid sequence, wherein a selected nucleic acid or amino acid sequence has at least about 70 or about 75% sequence identity as compared to a selected reference nucleic acid or amino acid sequence. More typically, the selected sequence and the reference sequence will have at least about 80, 81, 82, 83, 84 or even 85% sequence identity, and more preferably, at least about 86, 87, 88, 89, 90, 91, 92, 93, 94, or 95% sequence identity. More preferably still, highly homologous sequences often share greater than at least about 96, 97, 98, or 99% sequence identity between the selected sequence and the reference sequence to which it was compared.

The percentage of sequence identity may be calculated over the entire length of the sequences to be compared, or may be calculated by excluding small deletions or additions which total less than about 25% or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides.

When highly-homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988).

As used herein, the term “structural gene” is intended to generally describe a polynucleotide, such as a gene, that is expressed to produce an encoded peptide, polypeptide, protein, ribozyme, catalytic RNA molecule, or antisense molecule.

As used herein, the term “spheroid” refers to a three-dimensional spherical cellular aggregate culture model. Spheroids (e.g. hepatospheres) may better simulate a live cell's environmental conditions compared to a two-dimensional culture model, specifically with respect to reactions between cells.

The term “subject,” as used herein, describes an organism, including a mammal such as a human primate, to which treatment with one or more of the disclosed compositions may be provided. Mammalian species that may benefit from the disclosed treatment methods include, without limitation, humans, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like. The term “host” refers to any host organism that may receive one or more of the pharmaceutical compositions disclosed herein. Preferably, the subject is a vertebrate animal, which is intended to denote any animal species (and preferably, a mammalian species such as a human being). In certain embodiments, a “patient” refers to any animal host including without limitation any mammalian host. Preferably, the term refers to any mammalian host, the latter including but not limited to, human and non-human primates, bovines, canines, caprines, cavines, corvines, epines, equines, felines, hircines, lapines, leporines, lupines, murines, ovines, porcines, ranines, racines, vulpines, and the like, including livestock, zoological specimens, exotics, as well as companion animals, pets, and any animal under the care of a veterinary practitioner.

As used herein, the terms “terminal repeat” or “TR” mean a nucleic acid sequence derived from an AAV that is required in cis for replication and packaging of AAV.

“Transcriptional regulatory element” refers to a polynucleotide sequence that activates transcription alone or in combination with one or more other nucleic acid sequences. A transcriptional regulatory element may include, for example, one or more promoters, one or more response elements, one or more negative regulatory elements, one or more enhancers, or any combination thereof.

As used herein, a “transcription factor recognition site” and a “transcription factor binding site” refer to a polynucleotide sequence(s) or sequence motif(s) that are identified as being sites for the sequence-specific interaction of one or more transcription factors, frequently taking the form of direct protein-DNA binding. Typically, transcription factor binding sites may be identified by DNA footprinting, gel mobility shift assays, and the like, and/or may be predicted based on known consensus sequence motifs, or by other methods known to one of ordinary skill in the relevant molecular biological and virology arts.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis sequences that are necessary for efficient transcription and translation (e.g., polyadenylation site(s), mRNA stability controlling sequence(s), etc.

As used herein, the term “transformed cell” is intended to mean a host cell whose nucleic acid complement has been altered by the introduction of one or more exogenous polynucleotides into that cell.

As used herein, the term “transformation” is intended to generally describe a process of introducing an exogenous polynucleotide sequence (e.g., a viral particle, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

As used herein, the terms “treat,” “treating,” and “treatment” refer to the administration of a composition to reduce the frequency or severity of at least one sign or symptom of a disease, disorder or condition experienced by a subject. These terms embrace prophylactic administration, i.e., prior to the onset of clinical symptoms of a disease state so as to prevent any symptom or characteristic of the disease state. The disclosed compositions may be administered to a subject in an effective amount, that is, an amount capable of producing a desirable result. The desirable result will depend upon the active agent being administered. For example, an effective amount of a rAAV particle may be an amount of the particle that is capable of transferring a heterologous nucleic acid to a host organ, tissue, or cell. In some embodiments, the disease, disorder or condition is AAT, FAP, OTC Deficiency, or Wilson's Disease. Such treating need not be absolute to be deemed medically useful. As such, the terms “treatment,” “treat,” “treated,” or “treating” may refer to therapy, or the amelioration or reduction in the extent or severity of disease, disorder or condition, of one or more symptom thereof, whether before or after onset of the disease, disorder or condition.

As used herein, the term “vector” refers to a nucleic acid molecule capable of transporting another nucleic acid to which it has been linked, e.g., a plasmid. One type of preferred vector is an episome, i.e., a nucleic acid capable of extra-chromosomal replication. Preferred vectors are those capable of autonomous replication and/or expression of nucleic acids to which they are linked. An “rAAV nucleic acid vector” is a recombinant AAV-derived nucleic acid containing at least one terminal repeat (TR) sequence.

The use of “virion” is meant to describe a virus particle that contains a nucleic acid and a protein coat (capsid). An “rAAV virion” is a virion that includes nucleic acid sequences and/or proteins derived from a rAAV expression construct.

As used herein, the term “tropism” refers to the cells and/or tissues of a host which support growth of a particular serotype of AAV. Some serotypes may have a broad tissue tropism and can infect many types of cells and tissues. Other serotypes may infect primarily a single tissue or cell type.

As used herein, the term “variant” refers to a molecule (e.g. a capsid) having characteristics that deviate from what occurs in nature, e.g., a “variant” is at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type capsid. Variants of a protein molecule, e.g. a capsid, may contain modifications to the amino acid sequence (e.g., having 1, 2, 3, 4, 5, 6, 7, 8, 9, 10, 10-15, or 15-20 amino acid substitutions) relative to the wild type protein sequence, which arise from point mutations installed into the nucleic acid sequence encoding the capsid protein. These modifications include chemical modifications as well as truncations.

By a protein (e.g., a capsid protein) comprising an amino acid sequence having at least, for example, 95% “identity” to a query amino acid sequence, it is intended that the amino sequence of the subject amino acid molecule is identical to the query sequence except that the subject amino acid molecule sequence may include up to five amino acid alterations per each 100 amino acids of the query sequence. In other words, to obtain a capsid having an amino sequence at least 95% identical to a reference (query) sequence, up to 5% of the amino acids in the subject sequence may be inserted, deleted, or substituted with another nucleotide. These alterations of the reference sequence may occur at the N- or C-terminus of the reference sequence or anywhere between those positions, interspersed either individually among amino acids in the reference sequence or in one or more contiguous groups within the reference sequence.

As a practical matter, whether any particular amino acid molecule is at least 80%, 85%, 90%, 95%, 96%, 97%, 98%, or 99% identical to, for instance, the amino acid sequence of a capsid protein, can be determined conventionally using known computer programs. A preferred method for determining the best overall match between a query sequence (e.g., a sequence of the present disclosure) and a subject sequence, also referred to as a global sequence alignment, can be determined using the FASTDB or blastn computer program based on the algorithm of Brutlag et al. (Comp. App. Biosci. 6:237-245 (1990)). In a sequence alignment the query and subject sequences are either amino acid sequence or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. Whether a nucleotide is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present disclosure. For subject sequences truncated at the N- or C-terminus, relative to the query sequence, the percent identity is corrected by calculating the number of nucleotides of the query sequence that are positioned N- or C-terminus to the query sequence, which are not matched/aligned with a corresponding subject nucleotide, as a percent of the total bases of the query sequence.

### EXAMPLES

The following examples are included to demonstrate illustrative embodiments of the invention. It should be appreciated by those of ordinary skill in the art that the techniques disclosed in these examples represent techniques discovered to function well in the practice of the invention, and thus can be considered to constitute preferred modes for its practice. However, those of ordinary skill in the art should, in light of the present disclosure appreciate that many changes can be made in the specific embodiments which are disclosed and still obtain a like or similar result without departing from the spirit and scope of the invention.

### Example 1

### Step 1: Sub-Libraries Assembly.

Using pITR3-R3C3-AatII as a template, the following ten PCR reactions were conducted:

| A3CL-A (VRs-1, V, VI) : | PCR |
|---|---|
| Primers | fragment size |
| 1. A3CL-F + A3CL-A1R (before VR-I) | 86 bp |
| 2. A3CL-A1F + A3CL-A2R (VR-1 to most of VR-V) | 747 bp |
| 3. A3CL-A2F + A3CL-A3R (part of VR-V to VR-VI) | 136 bp |
| 4. A3CL-A3F + A3CL-R (after VR-VI) | 281 bp |
| A3CL-B (VR-IV): | PCR |
| Primers | fragment size |
| 5. A3CL-F + A3CL-B1R (before VR-IV) | 647 bp |
| 6. A3CL-B1F + A3CL-R (VR-IV to end) | 556 bp |
| A3CL-C (VR-VII): | PCR |
| Primers | fragment size |
| 7. A3CL-F + A3CL-C1R (before VR-VII) | 935 bp |
| 8. A3CL-C1F + A3CL-R (VR-VII to end) | 266 bp |
| A3CL-D (VR-VIII): | PCR |
| Primers | fragment size |
| 9. A3CL-F + A3CL-D1R (before VR-VIII) | 1055 bp |
| 10. A3CL-D1F + A3CL-R (VR-VIII to end) | 147 bp |

The respective PCR fragments were eluted from the agarose gel, mixed at equimolar ratios as indicated above for sub-libraries A, B, C, and D, and subjected to 15 cycles of overlap extension (OE) without primers, followed by 20 cycles of PCR using A3CL-F forward and A3CL-R reverse primers. The resulting fragments of 1140 bp for each of the A (I+V+VI), B (IV), C (VII), or D (VIII) sub-libraries were purified on agarose gel and eluted in small volume H2O. Using isothermal DNA assembly protocol, the respective fragments were individually sub-cloned into gel-purified pTR3-R3C3-AatII digested with AatII+ApaI. Four plasmid libraries A, B, C, and D, incorporating the respective VRs were derived. The estimated plasmid libraries' complexities were the following: A—4.4×10⁷; B—1.7×10⁷; C—1×10⁸; D—1×10⁸.

### Step 2: Pre-Selecting Structurally Compatible Parent Viral Libraries.

Using plasmid libraries from Step 1, viral sub-libraries A, B, C, and D were packaged, AAV virus from each preparation was purified using iodixanol density gradients, and the viral DNAs were isolated. Next, using viral DNAs as templates, the following PCR reactions were conducted:
1. VR-I, primers A3CL-F+VR-I_IV-R, template A, size 644 bp.
2. VR-IV, primers VR-I_IV-F+VR-IV_V-R, template B, size 145 bp.
3. VR-V+VI, primers VR-IV_V-F+VR-VI_VII-R, template A, size 194 bp.
4. VR-VII, primers VR-VI_VR-VII-F+A3CL-R, template C, size 274 bp.
5. VR-VIII, primers A3CL-F and A3CL-R, template D, size 1140 bp.

The respective PCR fragments were gel-purified and used as the templates in the OE/PCR to derive two PCR fragments, each of 1140 bp: A+B+C (VR-I, IV, V, VI, VII) and D (VR-VIII).

### Step 3: Packaging Master Libraries.

Using isothermal DNA assembly protocol, the respective fragments were individually sub-cloned into gel-purified pTR3-R3C3-AatII digested with AatII+ApaI. The estimated plasmid library A+B+C complexity was 2.5×10⁷, plasmid library D complexity was 4×10⁷. Using these plasmid libraries, two final master viral libraries were packaged: ABC, with the titer of 5.7×10¹² DNase resistant particles per milliliter (DRP/ml), and D, with the titer of 8.7×10¹² DRP/ml. The assembly flowchart is shown in FIG. 5.

TABLE 1
Theoretical (calculated) complexities of A3CL for individual
VRs and combinations of VRs. The VRs and VRs combinations
constructed as sub-libraries are shown in bold font.

| VR | Complexity |
|---|---|
| I | 72 |
| IV | 2.1 × 10⁶ |
| V | 27,648 |
| VI | 144 |
| VII | 4 × 10⁷ |
| VIII | 5.44 × 10⁸ |
| I + V | 1.99 × 10⁶ |
| I + VI | 1.04 × 10⁴ |
| I + V + VI | 2.87 × 10⁸ |
| V + VI | 3.98 × 10⁶ |
| I + IV + V + VI + VII | 2.37 × 10²² |
| I + IV + V + VI + VII + VIII | 1.29 × 10³¹ |

TABLE 2
Theoretical (calculated) complexities of constructed
sub-libraries A, B, C and D.

| Sub-library | VRs | Complexity |
|---|---|---|
| A3CL-A | I-V-VI | 2.9 × 10⁸ |
| A3CL-B | IV | 2 × 10⁶ |
| A3CL-C | VII | 4 × 10⁷ |
| A3CL-D | VIII | 5.4 × 10⁸ |

TABLE 3
Synthetic oligonucleotides used to assemble the AAV3B capsid library

| Name | Sequence |
|---|---|
| A3CL-F | GGCTGGGCGACAGAGTCATC (SEQ ID NO: 20) |
| A3CL-A1R | GCTGGAGATTTGCTTGTAGAGATG (SEQ ID NO: 21) |
| A3CL-A1F | CATCTCTACAAGCAAATCTCCAGCVVMDCAGGAGCTASCAACGACAACCACTACTTTGGC (SEQ ID |
| | NO: 22) |
| A3CL-A2R | CCAAGGAAASTYACTGTTGTTGTTSYSGBYGKVGRYTKTTGAAAGTCTCTGTTGCC |
| | (SEQ ID NO: 23) |
| A3CL-A2F | AACAACAACAGTRASTTTCCTTGGMCAGCGGCCAGCAMATATCATCTCAATG |
| | (SEQ ID NO: 24) |
| A3CL-A3R | GATTGCCGTGCATAGGGAAAAATYTSYCSKYATCGTCCYYGTGACTGGCCATAGCTGG |
| | (SEQ ID NO: 25) |
| A3CL-A3F | ATTTTTCCCTATGCACGGCAATC (SEQ ID NO: 26) |
| A3CL-R | CATCCGTGTGAGGAATCTTTGC (SEQ ID NO: 27) |
| A3CL-B1R | TTGCGTTCTGTTCAGGTAGTACAGA (SEQ ID NO: 28) |
| A3CL-B1F | CTGTACTACCTGAACAGAACGCAARGCAMCVCNRGCGGAACARCCRVCMHSMRSVVSCTG |
| | VNGTTTAGCCAGGCTGGGCC (SEQ ID NO: 29) |
| A3CL-C1R | TTTGCCAAATATTAGATTGCC (SEQ ID NO: 30) |
| A3CL-C1F | CGGCAATCTAATATTTGGCAAASAARRCRSCRVSRVARVCRATRYCGMSDWCGRSVRS |
| | GTAATGATTACGGATGAAGAAG (SEQ ID NO: 31) |
| A3CL-D1R | CTGCAAGTTATTTGCCACAGTTC(SEQ ID NO: 32) |
| A3CL-D1F | GAACTGTGGCAAATAACTTGCAGRVSVVSMRSRVCVVSCCCACGDHTVVSRNSGTC |
| | VMSCATCAGGGGGCCTTACCTG (SEQ ID NO: 33) |
| VR-I_IV-F | CAGTATCTGTACTACCTGAACAGAACGC(SEQ ID NO: 34) |
| VR-I_IV-R | GCGTTCTGTTCAGGTAGTACAGATACTG (SEQ ID NO: 35) |
| VR-IV_V-F | CCTGGGCCCTGCTACCGGCAACAGAG (SEQ ID NO: 36) |
| VR-IV_V-R | CTCTGTTGCCGGTAGCAGGGCCCAGG (SEQ ID NO: 37) |
| VR-VI_VII-F | CCCTATGCACGGCAATCTAATATTTGGC (SEQ ID NO: 38) |
| VR-VI_VII-R | GCCAAATATTAGATTGCCGTGCATAGGG (SEQ ID NO: 39) |

### Next Generation (NGS) Sequencing.

Number of sequences processed: 1817050
Number of distinct sequences (complexity): 1708473 (0.94)

Copy number distribution:

| Copy number | Number of sequences |
|---|---|
| 1 | 1603700 |
| 2 | 101430 |
| 3 | 3257 |
| 4 | 83 |
| 5 | 2 |
| 377 | 1 |

TABLE 4
Examples of the most representative variants within VRs IV, V, VI, and VII
from the master viral library ABC as deduced from the NGS sequencing (the dots in each of
sequences 1-86 corresponding to SEQ ID NOs: 44 and 45-129 below represent amino acid
residues that are identical to those listed in wild type as shown below).

| | 450 | 491 | 528 | 546 | cn | % |
|---|---|---|---|---|---|---|
| Wild type | GTTSGTTNQSRLL | KTANDNNNSNFPWTAASK | KDDEEK | EGTTASNAELDN | | |
| 1 | ............. | .................. | ...... | ............ | 377 | 0.0 |
| 2 | .N.G...SP...R | .IYDR............T | G..TGR | Q..GEG.V.VGK | 5 | 0.0 |
| 3 | S......G.RK.A | .AYGH....D...P...T | G...DR | QDSGENDVAIGR | 5 | 0.0 |
| 4 | ..A....AN.N.K | ..YS.............. | G..DDR | ...DGA.V.I.R | 4 | 0.0 |
| 5 | ..P...AAHKT.E | ..SAE............. | G..AGR | .DAEGGD.AIGG | 4 | 0.0 |
| 6 | S.AG..AT.KA.T | .VHAH............. | E..TGR | QDA.R..VAFEE | 4 | 0.0 |
| 7 | .NP....GLRG.T | T.D.E........P.... | ...AG. | Q..DGN.IAFGE | 4 | 0.0 |
| 8 | .N.....SKRP.M | T...E............T | E..N.R | .DAKGTDT.F.R | 4 | 0.0 |
| 9 | SNA....GIHQ.K | TAPDR....E........ | E..NGR | QNGATADT.VER | 4 | 0.0 |
| 10 | SN.G...AMRE.E | .AP......K.......T | E..TG. | .S.AETDV.DGR | 4 | 0.0 |
| 11 | SNA...AGLQ..K | .IPDQ............. | ...NG. | QSGG.ADIDNG. | 4 | 0.0 |
| 12 | .NP....APH... | TIH.G........P...T | ...DGR | QDGGT..IDI.G | 4 | 0.0 |
| 13 | ..P....DLRE.A | .IP..............T | E...DR | ............ | 4 | 0.0 |
| 14 | SNP...A.PRT.M | .IDAH....E...P...T | E..NG. | QSS.TGDV.D.D | 4 | 0.0 |
| 15 | .NA....DTK..T | .ASGG............T | ...DD. | ..SNRDD..V.R | 4 | 0.0 |
| 16 | .N.G...DIR..R | ..HSE....E.......T | ...N.R | QD.RETDVAI.R | 4 | 0.0 |
| 17 | .NA...AGMRE.M | .A..H............. | E...D. | .SGS.DDVAIGR | 4 | 0.0 |
| 18 | SN.G..ATPKQ.Q | .ASAH....E........ | .....R | ..S.RNDIANEH | 4 | 0.0 |
| 19 | SN.G..A.IKE.T | ..S..............T | R..ND. | QSASKNDI.YEQ | 4 | 0.0 |
| 20 | SNAG...SNRE.R | T.SSQ............T | R..DDR | QDAGGNDV.VGD | 4 | 0.0 |
| 21 | SN....ATT.A.K | ..YGH............. | G..T.. | Q.GS.N.V.VES | 4 | 0.0 |
| 22 | SNAG..AATN... | .IYDR............T | R...D. | ..GEKG.VDI.R | 4 | 0.0 |
| 23 | S.P....ATKG.T | TAHTG............. | G..DG. | ..S..TDVAIGS | 4 | 0.0 |
| 24 | .N.G...DLR..M | T.D.H....E...P...T | G..KGR | .NGAKNDIAFEG | 4 | 0.0 |
| 25 | S......TLKA.Q | .IP.R............T | G...DR | .NSKGA.T.I.E | 4 | 0.0 |
| 26 | .......DPKD.V | T.HG.....D.......T | ...DD. | .D.A.D.V.FGR | 4 | 0.0 |
| 27 | S.AG...TIKD.V | .VPD.....K........ | E..D.R | QDSG.T.V.FGR | 4 | 0.0 |
| 28 | ...G...TMRK.G | .VYGG............T | E..A.R | QSSGRNDV.YGD | 4 | 0.0 |
| 29 | .N.G..ASTR..T | .IPDQ........P.... | E...GR | QSAEKGDI.YGR | 4 | 0.0 |
| 30 | .N.....ATHT.A | .IHSR....D...P.... | E..AG. | Q.A..G.IDVEQ | 4 | 0.0 |
| 31 | SNPG...SIRG.Q | TIP.R........P...T | R..TD. | Q.GG.G.TDF.H | 4 | 0.0 |
| 32 | S.....AAPRG.V | TVYGH....E........ | ....GR | ..AG...VAIEE | 4 | 0.0 |
| 33 | .NA...ATKQG.M | .VP.Q....D........ | ...DDR | QSSDKN...D.S | 4 | 0.0 |
| 34 | SNAG..ATT.Q.R | TAPAE........P...T | R..ADR | .SGRGD.VDFEK | 4 | 0.0 |
| 35 | SN.G..AGIRA.Q | .VDTG....D.......T | E..T.. | .NSARND.DIGR | 4 | 0.0 |
| 36 | .NA...AA.NG.R | .IP.E....K.......T | ....G. | .SSSGDD..FGG | 4 | 0.0 |
| 37 | SN....AGPQQ.R | ..HAQ............. | E..TG. | ..AR.NDIAF.Q | 4 | 0.0 |
| 38 | S.P....SMRT.E | .APAR....E.......T | R..AG. | Q.SRENDT.F.G | 4 | 0.0 |
| 39 | S.AG...ALKG.K | TI.DH....E...P...T | R..K.. | .DS.GA.IAD.R | 4 | 0.0 |
| 40 | S.P...ASTRT.M | ..H.H....E.......T | ....D. | ...E.T.VAIGG | 4 | 0.0 |
| 41 | .NPG....NQA.R | .IHGQ....D.......T | R..ND. | ..SARGDVAYEK | 4 | 0.0 |
| 42 | SNA....DTRE.V | TI.D.....E.......T | R..TD. | Q.SAGADV.VEK | 4 | 0.0 |
| 43 | SNPG....LRE.R | TIHTE....E.......T | R..KDR | Q.GGGT.V.IGS | 4 | 0.0 |
| 44 | ...G..A.NNT.. | .I.SG............T | ...KGR | ..AEKNDTAVG. | 4 | 0.0 |
| 45 | .......DKQQ.M | ..H.G....D........ | E..TG. | QSAEGN.VAY.G | 4 | 0.0 |
| 46 | ..AG..ATL.T.V | .ISAG....D........ | G..NG. | QNS...DVAI.G | 4 | 0.0 |
| 47 | SN....AGLRT.T | .ADA.....D...P...T | G..NG. | .DASGN.V.DGR | 4 | 0.0 |
| 48 | SNA...ATP.T.R | ..DTH....E...P.... | R..NDR | ..ARG..IDVGD | 4 | 0.0 |
| 49 | S.A...ASLRA.M | .VP.R............T | G..ND. | .NAR..D..V.R | 4 | 0.0 |
| 50 | ..A...ATTKG.. | .ISTQ............. | E..AD. | Q.GETD.VDVGD | 4 | 0.0 |
| 51 | ..A...AALKQ.A | .ADS.............T | E..ADR | Q.GETG.I.Y.G | 4 | 0.0 |
| 52 | .NA...ATT.N.M | .ADDR............T | ....DR | Q.AKR.DTAVEE | 4 | 0.0 |
| 53 | ..AG..A.MKD.R | T..SE....D.......T | E..KD. | ..ANGGDVAIGQ | 4 | 0.0 |
| 54 | S.PG...TIRD.K | TVST.....D...P...T | ...DDR | ..SGRN.VAVEE | 4 | 0.0 |
| 55 | S.P...A.INT.R | ..P.R............T | E...GR | QSA.KDDVDIGG | 4 | 0.0 |
| 56 | .N.G..AGLQK.M | ..HGG........P...T | ...DG. | QSSRGNDVAV.D | 4 | 0.0 |
| 57 | .N.....TPRT.A | .IPSH....E........ | ...... | Q.SNG..I.FGS | 4 | 0.0 |
| 58 | SNAG..AGLRQ.T | .APAE....D.......T | E..AG. | ..GGGA.IAVEE | 4 | 0.0 |
| 59 | ..A...AAK.T.V | .ISTR............T | E | ...SKNDV.VE. | 4 | 0.0 |
| 60 | .......TTR..M | .TYGG............T | E..AGR | Q..ATA.V.VES | 4 | 0.0 |
| 61 | ..AG..AGMRE.A | TIYTG............. | ....GR | ..SSTGD.DVGR | 4 | 0.0 |
| 62 | S..G..A.PKE.R | TA..H....E.......T | E...D. | Q.AGE..VAI.G | 4 | 0.0 |
| 63 | .......GT.T.R | ..DTG............. | G....R | ..AGTAD.AV.G | 4 | 0.0 |
| 64 | .NAG....KRD.. | TAYTR....D.......T | ...D.. | Q..GKTD.DNGG | 4 | 0.0 |
| 65 | .NA....DMKH.T | .ISDR........P...T | R..N.R | QS.RGG...I.G | 4 | 0.0 |
| 66 | SN.G..ADLRD.. | TIPTQ....E........ | ...NDR | QSAK.NDV.V.R | 4 | 0.0 |
| 67 | S.A....ATQQ.V | T.DSQ....E.......T | R..NDR | .NAEGG.V.IGQ | 4 | 0.0 |
| 68 | .NAG...ANKT.M | .I.AH....E.......T | R..DG. | QDSS.D.I.YGK | 4 | 0.0 |
| 69 | .NAG...GTKE.R | TI..E............T | ...D.. | .DAKRN.VDY.G | 4 | 0.0 |
| 70 | SNP....GK.S.K | ..S.E............. | ...NG. | .DSR.GD.DFEK | 4 | 0.0 |
| 71 | .N....ASIRQ.Q | ..PDG....K........ | R..NGR | ..S.EG.I.IEG | 4 | 0.0 |
| 72 | .N.G...TL.A.G | TAHTQ............. | R..ND. | QSS.GGDTAF.G | 4 | 0.0 |
| 73 | SNP....TTQ..Q | ..D......D...P...T | R..TDR | ..S.GGD..IER | 4 | 0.0 |
| 74 | SNA....TMRK.G | TISSG........P.... | G..N.. | QDSSENDVADER | 4 | 0.0 |
| 75 | S.AG..ATMQ..M | T.DTG............T | R..N.R | Q.GEGGDI.D.R | 4 | 0.0 |
| 76 | ..AG..ATTRD.Q | T.DDH....D........ | ...NGR | Q.GRGA.TAYEG | 4 | 0.0 |
| 77 | ...G..AAM.A.R | T.DDG....K...P.... | ...... | .DGGT...AIGD | 4 | 0.0 |
| 78 | .N.....TNRE.M | .IP..............T | R...D. | .D.GRADV.VGR | 4 | 0.0 |
| 79 | SNAG..ADKQD.V | TAHSE............. | E..DDR | Q.AAGGDI.VGS | 4 | 0.0 |
| 80 | .NA...AATHE.. | T.HDH....D........ | R..A.R | ..GAK.DVDFGS | 4 | 0.0 |
| 81 | SNA...ADTRH.M | T.PGE....D...P...T | G..TG. | Q.SATTDI.YGE | 4 | 0.0 |
| 82 | S.....NA..K.Q | .IH.R....D.......T | R..DDR | Q.AEG.DVAVGD | 4 | 0.0 |
| 83 | .NP...AD.RA.Q | .IPTG....D.......T | R..T.. | Q..GG.DI.IGG | 4 | 0.0 |
| 84 | SNA...AGLNA.K | .AYTH....D.......T | G..D.R | .NAK.G..AI.G | 4 | 0.0 |
| 85 | .NP.....LQ..M | .IDDQ............P | ....D. | .SGGTADVAV.K | 4 | 0.0 |
| 86 | ......ASIQ..Q | ..YA.....E.......T | ....D. | .SAAG.DT.V.G | 4 | 0.0 |

Calculated plasmid library complexity based on colony count (2.5×10⁷) and NGS sequencing (0.94 of unique sequences) is 2.35×10⁷. wt AAV3 contamination is 0.02%.

| | |
|---|---|
| Q5 PCR: | |
| 50 ul: | 10 μl 5xB Q5 |
| | 0.4 μl 25 mM dNTPs |
| | 2.5 μl F |
| | 2.5 μl R |
| | 1 μl (1 ng) pITR3-R3C3-Aatll |
| | 0.5 μl Q5 Pol |
| | H₂O up to 50 μl |
| 98° C. 30 sec | 30 cycles |
| 98° C. 10sec | |
| 65° C. 20 sec | |
| 72° C. 30 sec | |
| 72° C. 2 min | |

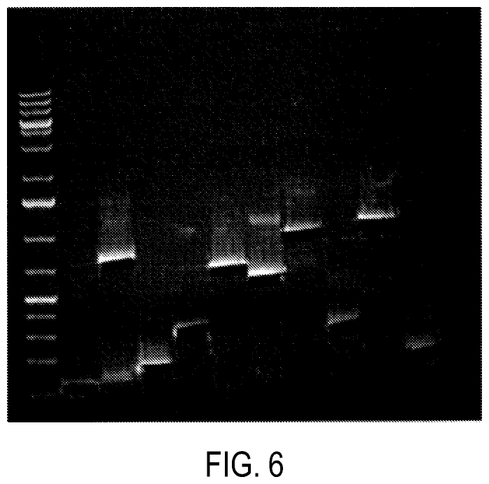

See FIG. 6.

TABLE 5

| | | | Conc. | | μl/5 × l0⁹ |
|---|---|---|---|---|---|
| | Fragment | Size (bp) | (μg/ml) | nM | copies |
| A | 1 | 86 | 10.4 | 186 | 2.2 |
| I + V + VI | 2 | 747 | 33.3 | 69 | 6 |
| | 3 | 136 | 13.2 | 149 | 2.8 |
| | 4 | 281 | 35.4 | 194 | 2.1 |
| B | 5 | 647 | 48 | 114 | 3.6 |
| IV | 6 | 556 | 29.4 | 81 | 5.1 |
| C | 7 | 935 | 22.5 | 37 | 11.2 |
| VII | 8 | 266 | 11.2 | 65 | 6.4 |
| D | 9 | 1055 | 38.8 | 57 | 7.3 |
| VIII | 10 | 147 | 35.9 | 376 | 1.1 |

TABLE 6
OE Q5 PCR

| | | | | | | | | 0 | xB | NTP | 3CL-F | 3CL-R | 5 | ₂O |
|---|---|---|---|---|---|---|---|---|---|---|---|---|---|---|
| .2 | .8 | .1 | | | | | | | 0 | .4 | .5 | .5 | .5 | 6 |
| | | | .6 | .1 | | | | | 0 | .4 | .5 | .5 | .5 | 0.4 |
| | | | | | 1.2 | .4 | | | 0 | .4 | .5 | .5 | .5 | 1.5 |
| | | | | | | | .3 | .1 | 0 | .4 | .5 | .5 | .5 | 0.7 |

1. Assays A, B, C, and D are assembled without primers, substituting H₂O for the primers' volumes (5 μl) and subjected to the following overlap extension:

| | |
|---|---|
| 98° C. 30 sec | 15 cycles |
| 98° C. 10 sec | |
| 65° C. 20 sec | |
| 72° C. 60 sec | |
| 72° C. 2 min | |

2. 40 μl each A, B, C, and D from Step 1 transferred to 10 μl containing:

| | X5 |
|---|---|
| 2.5 μl A3CL-F | 12.5 |
| 2.5 μl A3CL-R | 12.5 |
| 2 μl 5xB Q5 | 10 |
| 0.08 μl dNTPs | 0.4 |
| 0.1 μl Q5 | 0.5 |
| 2.82 μl H₂O | 14.1 |

Assays are subjected to the following PCRs:

| | |
|---|---|
| 98° C. 30 sec | 20 cycles |
| 98° C. 10 sec | |
| 59° C. 20 sec | |
| 72° C. 60 sec | |
| 72° C. 2 min | |

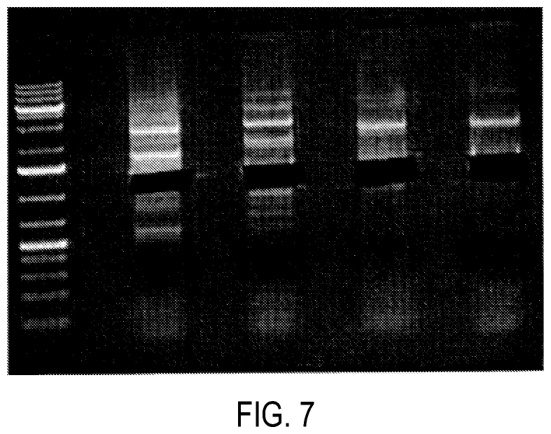

See FIG. 7: Eluted in 50 μl each A, B, C, or D; pTR3-R3C3-AatII/AatII+ApaI eluted in 75

TABLE 7

| | | | | | | pmoles/40 |
|---|---|---|---|---|---|---|
| | | | | | μl/40 μl | μl assay |
| | Size (bp) | Conc. (ng/μl) | Molarity (nM) | Conc. (pmoles/μl) | assay | (3:1) |
| A | 1091 | 47.5 | 67 | 0.067 | 2.1 | 0.144 |
| | | | | | (100 ng) | |
| B | 1091 | 58.3 | 82 | 0.082 | 1.8 | 0.144 |
| | | | | | (100 ng) | |
| C | 1091 | 49.9 | 70 | 0.07 | 2.1 | 0.144 |
| | | | | | (100 ng) | |
| D | 1091 | 60.9 | 86 | 0.086 | 1.7 | 0.144 |
| | | | | | (100 ng) | |
| pITR3-R3C3- | 6594 | 54.3 | 13 | 0.013 | 3.7 | 0.048 |
| AatII AatII-ApaI | | | | | (200 ng) | |
| cut | | | | | | |

| IDA | | | | | | | |
|---|---|---|---|---|---|---|---|
| 40 μl assay: 20 μl 2xGibson Master Mix (NEB)+ | A | B | C | D | pITR3 | H₂O | |
| | 2.1 | | | | 3.7 | 14.2 | A |
| | | 1.8 | | | 3.7 | 14.5 | B |
| | | | 2.1 | | 3.7 | 14.2 | C |
| | | | | 1.7 | 3.7 | 14.6 | D |

### Large-Scale IDA for the Loop A

| | |
|---|---|
| 300 μl assay: | 150 μl 2 × Gibson Master Mix |
| | 27.6 μl pITR3-R3C3-Aatll Aatll-Apal cut (1.5 μg) |
| | 15.8 μl A (0.75 μg) |
| | 106.6 μl H₂O |

Incubated 2 h, 50° C., Zymo-purified, eluted in 100 μl H₂O, combined with 47.5 μl of A from the pilot IDA above. Total—1.7 μg of vector plasmid DNA.
Lucigen competent cells were prepared from 4 L LB, resuspended in 8.5 ml H₂O final volume. The cell density (10 μl in 3 ml H₂O) was A₅₅₀=0.79.
Combined DNA (147.5 μl) was mixed with the whole volume of competent cells and aliquoted (385 μl/aliquot, ˜10 ng plasmid DNA/50 μl competent cells) into electroporation cuvettes (total of ˜20, with outside tall electrodes) and zapped at 2.9 KV.
Cells were transferred into 1 L LB, incubated shaking at 37° C. for 1 h. Carbenicillin was added up 100 μg/ml, cell were grown at 30° C., o/n.
Total complexity from the large-scale IDA/transformation is 4.4×10⁷ clones.

### Repeat IDA for the Loop C

| | |
|---|---|
| 100 μl assay: | 50 μl 2 × Gibson Master Mix |
| | 9.25 μl plTR3-R3C3-Aatil Aatll-Apal cut (0.5 μg) |
| | 5.25 μl C (0.25 pg) |
| | 35.5 μl H₂O |
| Zymo, 50 μl H₂O. | |

Competent cells were prepared from 4 L LB (grown to A₅₅₀=0.6) and resuspended in a final volume 8 ml H₂O. The cell density (10 μl in 3 ml H₂O) was A₅₅₀=1.46.
180 ng vector with fragment B from the pilot IDA were electroporated with 1 ml of comp. cells, whereas 0.68 μg with fragment C—with 3 ml of cells.
After electroporation the complexity of B was ˜1.7×10⁷ (˜5 times over theoretical complexity), while C—1×10⁸ (˜2.5 times over theoretical complexity).

TABLE 8

| Pilot | | | | | Large-scale | | | |
|---|---|---|---|---|---|---|---|---|
| | A | B | C | D | A | B | C | D |
| Complexity total | 0.9 × 10⁵ | 0.7 × 10⁵ | 1.4 × 10⁵ | 0.9 × 10⁵ | 4.4 × 10⁷ | 1.7 × 10⁷ | 1 × 10⁸ | 1 × 10⁸ |
| Volume (μl) | 400 | 400 | 400 | 400 | 1000 | 100 | 500 | 1000 |
| Complexity/μl | 225 | 175 | 525 | 225 | 4.4 × 10⁴ | 1.7 × 10⁵ | 2 × 10⁵ | 10⁵ |
| DNA concentration | 342 | 220 | 241 | 334 | 1690 | 1100 | 2100 | 2000 |
| (ng/μl) | | | | | | | | |
| Copies/μl | 4.2 × 10¹⁰ | 2.6 × 10¹⁰ | 2.9 × 10¹⁰ | 4 × 10¹⁰ | 2 × 10¹¹ | 1.3 × 10¹¹ | 2.5 × 10¹¹ | 2.4 × 10¹¹ |
| Representation | 1.9 × 10⁸ | 1.5 × 10⁸ | 0.6 × 10⁸ | 1.8 × 10⁸ | 4.5 × 10⁶ | 7.6 × 10⁵ | 1.3 × 10⁶ | 2.4 × 10⁶ |
| (copies/variant/μl) | | | | | | | | |
| Dilution factor | 42.2 | 197.4 | 46.2 | 75 | | | | |
| Final DNA | | | | | | 0.55 | 1.1 | 1 |
| concentration after | | | | | | | | |
| mixing equal | | | | | | | | |
| volumes (μg/μl) | | | | | | | | |
| Viral DNA | | | | | 27.6 | 22.8 | 23.2 | 85.5 |
| concentration, 80 | | | | | | | | |
| μl (μg/ml) | | | | | | | | |
| Titer (copies/μl) | | | | | | 4.5 × 10⁹ | 4.6 × 10⁹ | 1.6 × 10¹⁰ |

Q5 PCR of viral DNA
Conditions, as above, except: 50 ng viral DNA/50 μl assay, 20 PCR cycles 5 μl out of 50

1. Loop I, primers A3CL-F+VR-I_IV-R, template A, size 644 bp

2. Loop IV, primers VR-I_IV-F+VR-IV_V-R, template B, size 145 bp

3. Loops V+VI, primers VR-IV_V-F+VR-VI_VII-R, templ. A, size 194 bp

4. Loop VII, primers VR-VI_VR-VII-F+A3CL-R, template C, size 274 bp

Remaining 45 μl were purified using preparative gel, all four gel cutouts were pooled in one tube and purified using one column, final volume 50 μl H₂O.

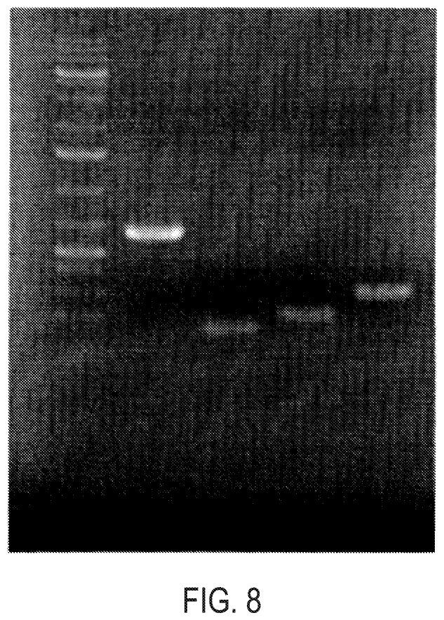

### See FIG. 8.

### Overlap Extension

Full-length fragment was assembled without primers, substituting H₂O for the primers' volumes (5 μl) and subjected to the following overlap extension:

| | |
|---|---|
| 50 μl: | 10 μl 5xB Q5 |
| | 0.4 μl 25 mM dNTPs |
| | 25 μl (out of 50 μl) individual overlap 4 fragments |
| | mix (p.12) |
| | 0.5 μl Q5 Pol |
| | 14.1 μl H₂O |
| 98° C. 30 sec | 15 cycles |
| 98° C. 10 sec | |
| 65° C. 20 sec | |
| 72° C. 60 sec | |
| 72° C. 2 min | |

After primer-less extension, the assay was split into 2×25 μl assays supplemented with A3CL-F, and A3CL-R primers, DNTPs, and fresh Q5, total volume 50 μl each.
Assays are subjected to the following PCRs:

| | |
|---|---|
| 98° C. 30 sec | 20 cycles |
| 98° C. 10 sec | |
| 59° C. 20 sec | |
| 72° C. 60 sec | |
| 72° C. 2 min | |

ABC fragment was eluted in 50 concentration 60 ng/(0.085 pmoles/μl).
D fragment was eluted in 50 concentration 46 ng/μl (0.065 pmoles/μl).

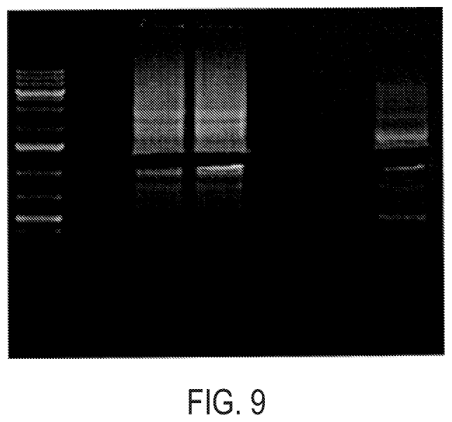

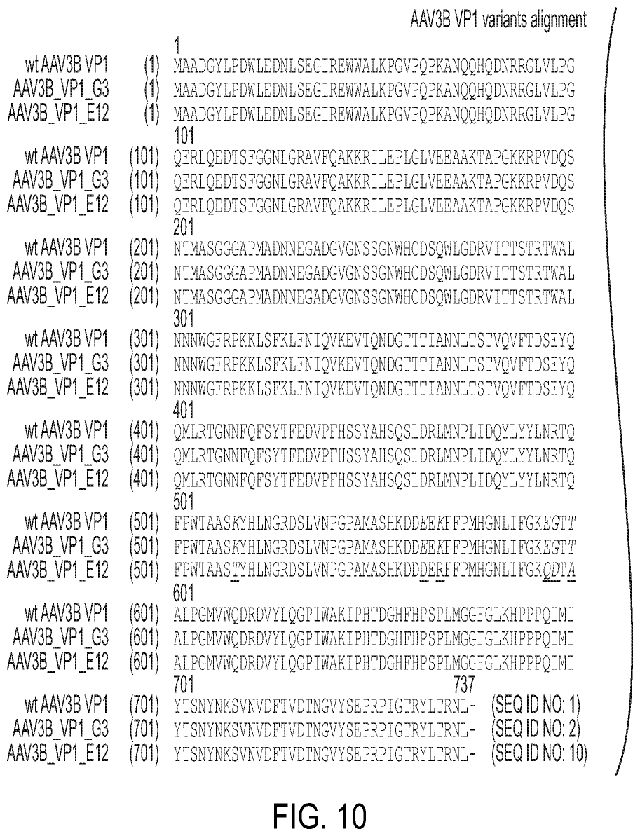

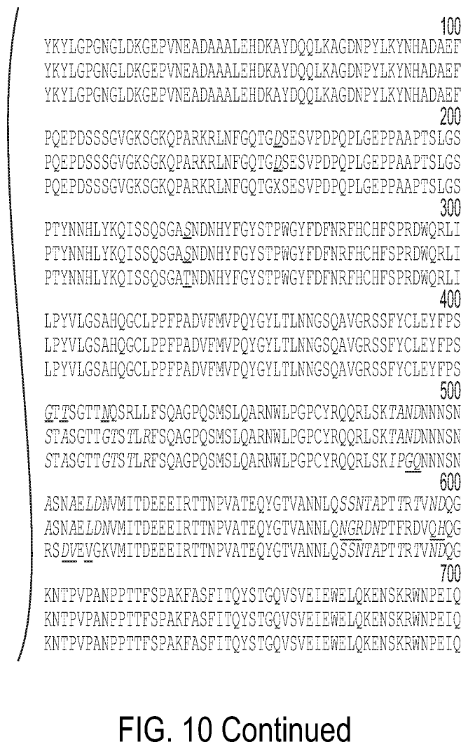

See FIG. 9.

### IDA Using NE Builder Master Mix

Total volume—200 μl, plasmid 1.5 μg (0.348 pmoles), insert—0.5 μg (0.7 pmoles), total DNA amount ˜1 mole/2000 assay.
Reaction 60 min @ 50° C. Lucigen electrocompetent E. coli cells, 8 ml, final density 0.8 A550. Library's complexity 2.5×10⁷.

### Example 2

In Vivo Iterative Selection of AAV3B-G3 and AAV3B-E12

The AAV3B library was selected for variants with enhanced adaptive survival and proliferation in the humanized livers of a transgenic mouse strain, i.e., NSG-PiZ, which expresses a human PiZ allele at the SerpinA1 locus.

NSG-PiZ mice were crossed with the NOD-SCID-gamma chain knockout (NSG) strain engrafted with human hepatocytes to create human liver xenografts as previously described in Borel F, et al. Mol Ther. 2017; 25(11): 2477-2489, herein incorporated by reference. A million human hepatocytes (Bioreclamation IVT) dispersed in 50 μL of Hanks Balanced Saline solution was injected into the inferior pole of the spleen of mice using a 25-gauge needle connected to a ⅓-ml syringe, with and without Partial liver hepatectomy. The degree of success of engraftment was evaluated by Human Albumin ELISA Quantitation Set (E80-129, Bethyl laboratories) following each round of selection. The measured human albumin levels are shown in Table 9, below.

Mice were administered original master AAV3B library by tail vein injection. Mice from the first two rounds of selection (Rounds 1 and 2) were divided into two groups. A first group (Group 1) was administered adenovirus into the tail vein (2.0×10⁹ pfu/mice) two weeks post AAV3B library injection. A second group (Group 2) was not administered adenovirus (“w/o Adeno”). In all subsequent groups receiving adenovirus, the adenovirus was administered two weeks after injection of the library. The adenovirus used was Adenovirus MVB lot 063005MRP, 1.0×10¹¹ pfu/ml (UF).

Animals were sacrificed two days after adenovirus delivery to group 1. Liver tissue was harvested and flash frozen. Tissues were used for PCR amplification to create lysates for the next round(s) of selection.

### Round 1

All 4 mice were injected with master AAV3B library at 4.3×10¹¹ pfu/mouse, (VRs I, IV, V, VI, and VII, and VR VIII combined).

Group 1: two mice. Two weeks after library injection, mice were administered adenovirus in an amount of 2.0×10⁹ pfu/mouse, and hepatic tissue was collected 48 hours post adenovirus injection.

Group 2: two mice. Tissue was collected two weeks post library injection.

### Round 2

Group 1: three mice were injected with the library as screened from Round 1, Group 1 at 1.58×10¹¹ vg/mouse (200 μl of 7.9×10¹¹ vg/ml). Mice were administered adenovirus in an amount of 2.0×10⁹ pfu/mouse, and tissue was collected 48 hours post adenovirus injection.

Group 2: three mice were injected with the library as screened from Round 1, Group 2 at 2.6×10¹⁰ vg/mouse (200 μl of 1.3×10¹¹ vg/ml). Tissue was collected two weeks post library injection.

### Round 3

Group 1: three mice were injected with the library as screened from Round 2, Group 1 in an amount of 1.74×10¹¹ vg/mouse (200 μl of 8.7×10¹¹ vg/ml). Mice were administered adenovirus, and tissue was collected 48 hours post adenovirus injection.

Group 2: three mice were injected with the library as screened from Round 2, Group 2 in an amount of 2.52×10¹¹ vg/mouse (200 μl of 1.26×10¹² vg/ml). Tissue was collected two weeks post library injection.

### Round 4

Only one group of mice was used in this round. Three mice were injected with the library (5 μl/mouse) as screened from Round 3, Group 2 in an amount of 3.0×10¹² vg/ml. Mice were administered adenovirus, and tissue was collected 48 hours post adenovirus injection.

### Round 5

Only one group, 4 mice were injection with the library (5 μl/mouse) in an amount of 3.88×10¹² vg/ml, H4 variant).

Mice were administered adenovirus, and tissue was collected 48 hours post adenovirus injection.

TABLE 9
Serum human Albumin levels in Engrafted Mice

| AAV3B | number | ug/ml | Note |
|---|---|---|---|
| Round 1 | 726 | 1.8 | Adeno |
| | 756 | 1.2 | Adeno |
| | 754 | 1.6 | w/o Adeno |
| | 755 | 1.2 | w/o Adeno |
| Round 2 | 987 | 0.7 | Adeno |
| | 988 | 0.82 | Adeno |
| | 990 | 0.43 | Adeno |
| | 985 | 0.36 | w/o Adeno |
| | 986 | 0.44 | w/o Adeno |
| | 988 | 0.76 | w/o Adeno |
| Round 3 | 209 | 0.32 | Adeno |
| | 214 | 0.25 | Adeno |
| | 215 | 0.75 | Adeno |
| | 210 | 0.87 | w/o Adeno |
| | 211 | 0.27 | w/o Adeno |
| | 216 | 0.36 | w/o Adeno |
| Round 4 | 232 | 0.22 | Adeno |
| | 233 | 0.16 | Adeno |
| | 234 | 0.33 | Adeno |
| Round 5 | 288 | 0.39 | Adeno |
| | 289 | 0.76 | Adeno |
| | 290 | 0.27 | Adeno |
| | 291 | 0.58 | Adeno |

What is claimed is:

1. A variant recombinant adeno-associated virus (rAAV) serotype 3B (AAV3B) capsid protein comprising each of the following sets of sequences and/or substitutions:
(a) STX₄X₅GTTGTX₈X₉LX₁₀ (SEQ ID NO: 7) in variable region (VR) IV wherein X₄ is P or A; X₅ is S or G; X₈ is S or N; X₉ is T or G; and X₁₀ is K or R;
(b) X₁₁X₁₂X₁₃X₁₄NNNSNFPWTAASX₁₅ (SEQ ID NO: 16) in VR V, wherein X₁₁ is I or T; X₁₂ is A or P; X₁₃ is N, S or G; X₁₄ is D or Q; and X₁₅ is K or T;
(c) KDDX₁₆X₁₇X₁₈ (SEQ ID NO: 17) in VR VI, wherein X₁₆ is E or D; X₁₇ is E or D; and X₁₈ is K or R; and
(d) one of QSSNTAPTTRTVND (SEQ ID NO: 6) or QNGRDNPTFRDVQH (SEQ ID NO: 8) in VR VIII;
wherein X may be any amino acid.

2. The variant of claim 1 comprising one or more of (a) STASGTTGTSTLR (SEQ ID NO: 3) in VR IV, (b) IPGQNNNSNFPWTAAST (SEQ ID NO: 4) in VR V, (c) KDDDER (SEQ ID NO: 9) in VR VI, and (d) QSSNTAPTTRTVND (SEQ ID NO: 6) in VR VIII.

3. The variant of claim 1 or 2 further comprising GKQDTARSDVEVGK (SEQ ID NO: 5) in VR VII.

4. The variant of any one of claims 1-3 further comprising the substitution S267T.

5. The variant of claim 1 comprising one or more of (a) STASGTTGTSTLR (SEQ ID NO: 3) in VR IV and (d) QNGRDNPTFRDVQH (SEQ ID NO: 8) in VR VIII.

6. The variant of claim 1 or 5, wherein the capsid protein comprises an amino acid sequence that is at least 95% identical to the sequence set forth in SEQ ID NO: 2.

7. The variant of claim 1 or 5, wherein the capsid protein comprises the amino acid sequence set forth in SEQ ID NO: 2.

8. The variant of any one of claims 1-4, wherein the capsid protein comprises an amino acid sequence that is at least 95% identical to the sequence set forth in SEQ ID NO: 10.

9. The variant of any one of claims 1-4, wherein the capsid protein comprises the amino acid sequence set forth in SEQ ID NO: 10.

10. A recombinant AAV3B particle comprising the recombinant AAV capsid protein of any one of claims 1-9.

11. The recombinant AAV3B particle of claim 10, further comprising a nucleic acid comprising a transgene of interest.

12. The recombinant AAV3B particle of claim 11, wherein the transgene is SERPINA1.

13. The recombinant AAV3B particle of claim 11, wherein the transgene is TTR.

14. The recombinant AAV particle of any one of claims 10-13, wherein the nucleic acid is single stranded.

15. The recombinant AAV particle of any one of claims 10-13, wherein the nucleic acid is self-complementary.

16. A composition comprising a plurality of the variant recombinant AAV3B particle of any one of claims 10-15.

17. The composition of claim 16 further comprising a pharmaceutically acceptable carrier.

18. A method of transducing a hepatic cell with a transgene of interest, the method comprising providing to the hepatic cell the variant recombinant AAV particle of any one of claims 10-15 or the composition of claim 16 or 17.

19. A method of treating a disease or disorder comprising administering the variant recombinant AAV particle of any one of claims 10-15, or the composition of claim 16 or 17, to a subject in need thereof.

20. The method of claim 19, wherein the disease or disorder is Alpha-1 Antitrypsin Deficiency.

21. The method of claim 19, wherein the disease or disorder is Transthyretin-Related Familial Amyloid Polyneuropathy.

22. The method of any one of claims 19-22, wherein the step of administering provides about a 15%, a 30%, a 50%, a 100%, a 200%, a 300%, a 400%, a 500%, a 750%, or a 1000% increase in transduction of the transgene of interest in hepatic cells in the subject, relative to a wild-type recombinant AAV3B particle.

23. The method of any one of claims 19-22, wherein the subject is a primate.

24. The method of any one of claims 19-23, wherein the subject is human.

25. The method of claim 18, wherein the hepatic cell is a human hepatocyte.

26. The variant recombinant AAV particle of any one of claims 10-15, or the composition of claim 16 or 17, for use as a medicament.
